(19) United States
(12) Patent Application Publication
(10) Pub. No.: US 2025/0035645 A1
(43) Pub. Date: Jan. 30, 2025

(54) Detection of B Cell Antibody Responses in Patients with Haemophilia and Treatment Thereof

(71) Applicant: Cellular Technology Limited, Shaker Heights, OH (US)

(72) Inventors: Paul VIktor Lehmann, Moreland Hills, OH (US); Loral Showalter, Akron, OH (US)

(73) Assignee: Cellular Technology Limited, Shaker Heights, OH (US)

(21) Appl. No.: 18/358,456

(22) Filed: Jul. 25, 2023

(30) Foreign Application Priority Data
Jun. 24, 2022 (WO) PCT/US2022/035001

(51) Int. Cl. G01N 33/68; A61K 45/06; A61P 7/04; C07K 16/28

(52) U.S. Cl. CPC G01N 33/686; A61K 45/06; A61P 7/04; C07K 16/2887

(57) ABSTRACT

Embodiments of this disclosure include in vitro tests to detect clotting factor-specific antibodies produced by B cells from peripheral blood mononuclear cells (PBMCs) in patients with haemophilia that develop resistance to substitution therapy. Additional embodiments include therapeutic methods for treating patients with haemophilia who have been identified at risk of developing B cell mediated resistance to clotting factor-reconstitution (or substitution) therapy, or have developed resistance to such therapy, by selectively inactivating the B cells that produce clotting factor-specific antibodies, and monitoring the efficiency of the latter treatment by measuring the residual clotting factor-specific B cells in these subjects.

### PRIORITY CLAIM

This application claims is being filed under 35 U.S.C. 1.111 (a) claiming priority to U.S. Provisional Patent Application No. 63/238,991 filed 31 Aug. 2021 and PCT/US22/035001, originally filed 24 Jun. 2022, and corrected 28 Dec. 2022, both entitled Detection of B Cell Antibody Responses in Patients with Haemophilia and Treatment Thereof. These applications are fully incorporated herein by reference in their entirety.

### FIELD OF THE INVENTION

This invention relates generally to detection of clotting factor-specific B cells in blood of patients with haemophilia who are developing B cell mediated resistance to clotting factor replacement (or substitution) therapy. Particularly, this invention relates to methods and assays for detecting such B cells as diagnostic, prognostic or therapeutic biomarkers of therapy resistance that can develop in hemophilia. More particularly, this invention relates to in vitro detection in the blood of B lymphocyte antibodies that bind to clotting factors, and treatment of such haemophilia using high amounts of the deficient clotting factor.

### SEQUENCE LISTING

The Sequence Listing in ST.26, version 1.6, approved Nov. 25, 2022, format written in file CLTL-1045US2_ST26.xml, is filed under 37 C.F.R. 1.831-1.839, the sequence listing created Feb. 27, 2023, 26,671 bytes, machine format IBM-PC, MS-Windows operating system, is hereby incorporated by reference in its entirety and for all purposes.

### BACKGROUND

Haemophilia is a blood disorder that results in failure of a subject to form blood clots when injured. Hemophilic patients may have a genetic defect that prevents them from making coagulation (or clotting) factors. Such patients depend on continuous replacement therapy comprising injections of exogenous clotting factors, administered either as recombinant proteins, purified clotting factors, or serum containing the clotting factor.

A common side effect of either treatment is, however, that the hemophiliacs become resistant to therapy because they produce antibodies against the injected clotting factor molecules that in immunological terms are foreign antigens to them. For some unknown reason some hemophiliac patients develop an antibody response to clotting factors; some patients develop it readily and others develop over time. Antibodies produced by the patients can neutralize the injected clotting factor thus making therapy inefficient or out-right ineffective resulting in B cell mediated resistance to substitution therapy.

### SUMMARY

Although it is widely assumed that the resistance to substitution therapy that hemophilic patients develop is mediated by antibodies, it has heretofore been impossible to detect these antibodies in the serum of patients who are in early phases of developing resistance to therapy using standard methods such as enzyme-linked immuno-assays (ELISA).

A reason for the inability to detect the clotting factor-specific antibodies in the serum of haemophilia patients in early phases of their development of resistance to substitution therapy is that these antibodies can be bound and captured constantly by the administered clotting factor and the resulting immune complexes are eliminated in the liver and spleen.

Instead of the detection of circulating clotting factor-specific antibodies in serum, it is now possible to detect the underlying B cell response by detecting the clotting factor-specific B cells themselves. Such B cells can be detected after polyclonal B cell stimulation ex vivo in an “indirect B cell assay” (also known as “indirect B cell test”), or via their spontaneous production of antibody ex vivo, in a “direct B cell assay” (also known as “direct B cell test”) To detect such B cells, we either use ELISPOT or FluoroSpot methods that permit to establish the numbers of B cells that secrete clotting factor-specific antibodies ex vivo, or to detect such antibodies in culture supernatants of B cells ex vivo using ELISAs or similar methods.

For a “direct B-cell test,” we: a) coat a membrane suitable for ELISPOT or FluoroSpot assays (in our case, PVDF) with a clotting factor, b) obtain peripheral blood mononuclear cells (PBMCs, that contain B cells), c) place the PBMC sample onto the membrane in cell culture fluid, d) permitting the B cells to produce antibodies (e.g., IgG) against the clotting factor without polyclonal activator. Once antibodies are bound to the clotting factor antigen, the antibodies can be detected using an labeled antibody-specific antibody, such as an anti-IgG antibody with an enzymatic label. Alternatively, an anti-antibody can be labeled with a fluorescent label (e.g., using a FluoroSpot™ assay). These detection methods permit detection and quantification of the labeled antibodies on the membrane using optical detection methods.

In a variant of the assay, the “indirect B cell test”, the PBMC are first activated by adding a B cell polyclonal activator (typically R848 and IL2, alone, or a combination of the former with or without IL6, IL10 and ß-mercaptoethanol, thymus-independent antigens, bacterial polysaccharides, polymeric bacterial proteins, bacterial lipopolysaccharides, B-cell mitogens, lipopolysaccharide or unmethylated single-stranded DNA motifs (CpG oligonucleotides), TLR2 (Toll-like receptor 2) receptor ligands, TLR4 receptor ligands, TLR9 receptor ligands, TLR7 receptor ligands, TLR9 receptor ligands, TLR10 receptor ligands, CD40 ligand, pokeweed mitogen, B-cell activating factor (BAFF), CD21 activators, C4b and C3b stimulators) for the duration of 3-8 days before the cells are seeded into cell culture plates for assays.

Polyclonal activators stimulate B cells to proliferate and to differentiate into antibody secreting cells (ASC). Once the B cells produce antibodies to a clotting factor, those antibodies bind to the clotting factor that has been coated onto the membrane. Once bound, these clotting factor-specific antibodies produced by the ASC can be visualized using optical detection methods using detection molecules. Detection molecules can be anti-human immunoglobulin antibodies that are visualized via an enzymatic reaction that cleaves a precipitating substrate to produce a colored product (e.g., ELISPOT) or via a fluorescent tag (FluoroSpot).

In an alternative version of the ELISPOT or FluoroSpot approaches, the plate is coated with an antibody that is specific for human immunoglobulin (e.g. anti-kappa/lambda antibody) that will capture all antibodies produced by ASC irrespective of their specificity; adding labelled clotting factor antigen or antigen-specific detection antibodies permits then to identify the ASC that produce clotting factor-specific antibodies.

Alternatively to ELISPOT or FluoroSpot, the clotting factor-specific antibodies produced by the ASC in cell culture can be detected as soluble molecules in the PBMC culture supernatant using ELISA, protein array, or other methods suited for detection of soluble antigen-specific antibodies.

### BRIEF DESCRIPTION OF THE FIGURES

The patent or application file may contain at least one drawing executed in color. Copies of this patent or patent application publication with color drawing(s) will be provided by the Office upon request and payment of the necessary fec.

This invention is described with respect to specific embodiments thereof. Other features of this invention can be appreciated and understood with reference to the Figures, in which:

FIG. 1 depicts a diagram of an embodiment 100 of a B cell test showing an assay plate (not shown) with membrane 105 of this invention, having a clotting factor 115 coating the plate, with B cell antibodies 125 against the clotting factor binding to the clotting factor antigens 115. Also shown are anti-human immunoglobulin specific detection antibodies 135 binding to B cell-produced antibodies 125. Also shown are detection moieties 145 having an enzyme and substrate 150 attached to the detection antibodies.

Detection moieties can be either enzyme linked immunodetection moieties or fluorescent moieties.

FIGS. 2 and 3 depict schematically, direct B-cell test (FIG. 2) assays and indirect B-cell test (FIG. 3) of this invention.

FIG. 2 depicts a “direct B-cell test” 200 of this invention showing an ASC (activated B cell or plasma cell 215 that produces clotting factor-specific antibodies 210. Plasma cell 215 is contained in a sample of peripheral blood mononuclear cells (PBMCs) and placed in a culture well (at arrow 225). Clotting factor-specific antibodies 230 are then detected using ELISPOT or similar methods, and show image 235 having spots indicative of ASC that produce clotting factor-specific antibodies.

FIG. 3 depicts an “indirect B-cell test” 300 of this invention. Memory B-cell 305 recirculates in the bloodstream. Memory B-cell 305 is then drawn along with a sample of PBMCs, which are placed in a culture well. Then, polyclonal stimulation 310 of memory B-cell 305 is accomplished by exposure of the cells to interleukin-2 (IL-2), R-848, and 2-mercaptoethanol (2-ME or β-ME) or other B-cell polyclonal activator(s). Memory B-cell 305 is thus stimulated and becomes ASC or plasma cell 320, which produces antibodies 325. Clotting factor-specific antibodies 325 are then detected using ELISPOT, ELISA, Fluorospot™ or similar methods, and show image 330 having spots indicative of cells that produce clotting factor-specific antibodies 325.

### DETAILED DESCRIPTION

### Definitions

The following definitions are included for convenience. The meanings of the following terms are to be defined immediately below, unless such term(s) are defined specifically in the sections that follow.

The terms “Clotting factor” and “Coagulation factor” mean proteins that are involved in clotting or coagulating of blood. These terms are interchangeable. Representative clotting factors include Factor I, Factor II, Factor V, Factor VII, Factor VIII, Factor IX, Factor X, Factor XI, Factor XIII, von Willebrand Factor (VWF), and antithrombin III.

“Clotting Factor Antigen” means a full length-protein of a clotting factor or an immunogenic portion thereof, sufficient to bind to an anti-clotting factor antibody. Such antigens include different chains, alpha-chains beta-chains and gamma-chains.

“AP” means alkaline phosphatase.

“ASC” means antibody secreting cells.

“B-cells” means B-lymphocytes, including memory B-cells that do not produce antibodies unless stimulated with a B-cell polyclonal activator, and ASC that include plasmablasts, plasma cells, effector B-cells, and B cells that spontaneously produce antibodies against a clotting factor without B-cell polyclonal activator.

“ß-mercaptoethanol”, “2-mercaptoethanol”, “2-ME” and “β-ME” means the chemical compound with the formula HOCH₂CH₂SH. It can be used for the polyclonal stimulation or activation of B lymphocytes.

“Blood antibodies” means antibodies present in blood or serum, including antibodies against clotting factors produced in vivo through antigen-specific responses of white blood cells, in particular B lymphocytes.

The term “comprising” means the listed elements and other named or unnamed elements.

The term “consisting essentially of” means the listed elements and their equivalents.

The term “consisting of” means the listed elements and no others.

“DNA” means desoxyribonucleic acid.

“ELISA” means enzyme-linked immunosorbent assay.

“ELISPOT” means enzyme-linked immunosorbent spot technique.

“FITC” means fluorescein isothiocyanate.

“Haemophilia” and “Hemophilia” mean a disorder characterized by increased bleeding.

“HRP” means horseradish peroxidase.

“Ig” means immunoglobulin antibody.

“IL” means interleukin.

“PBMCs” means peripheral blood mononuclear cells.

“Polyclonal activators” (also known as “B cell polyclonal activators” or “B cell polyclonal stimulators”) are agents that stimulate proliferation of B cells and induce B cells to produce antibodies. They include CpG (bacterial DNA), an antigen of Staphylococcus aureaus Cowan, SAC, R848 being a TLR7/8 agonist that mimics contact of B cells to bacteria. Pokeweed mitogen (PWM), IL-2 (interleukin 2), IL-6, IL-10, and B-cell activating factor (BAFF) also known as tumor necrosis factor ligand superfamily member 13B (TNFL 13B), Anti-CD40 antibodies, and combinations thereof, including R848 plus IL-2.

“PBL” means peripheral blood lymphocytes.

“R848” means an imidazoquinoline compound with potent anti-viral activity. This low molecular weight synthetic molecule activates immune cells via the TLR7/TLR8 MyD88-dependent signaling pathway. Recently, R848 was shown to trigger NF-κB activation in cells expressing murine TLR8 when combined with poly(dT). It is used for the polyclonal stimulation of B lymphocytes.

The term “Substitution Therapy” and “Replacement Therapy” mean treatment for haemophilia caused by deficiency of a clotting factor by administering exogenous clotting factor.

“VWF” means von Willebrand Factor.

### Haemophilia

Haemophilia is an inherited genetic disorder of certain clotting factors leading to a bleeding disorder (increased bleeding). Symptoms may include unexplained bleeding, pain, swelling of joints, large and deep bruises, bleeding gums, blood in urine or stool, nose bleeds, and irritability. People afflicted by Factor XI deficiency may have difficulty stopping the flow of blood following dental extractions, trauma or surgery. Women with factor XI deficiency may also experience heavy menstrual periods or heavy postpartum bleeding. Within affected people and their families, highly variable bleeding patterns occur, and bleeding risk cannot be predicted by the level of factor XI (a clotting factor) in the blood. Although the condition can affect people of all heritages, it is most common in people of Ashkenazi Jewish descent. Most cases of factor XI deficiency are inherited and caused by changes (mutations) in the F11 gene. In most cases the condition is inherited in an autosomal recessive manner however, it may follow an autosomal dominant pattern in some families. Treatment is often only recommended during periods of high bleeding risk (i.e. surgery) and may include fresh frozen plasma and/or antifibrinolytics (medications that improve blood clotting). Factor XI concentrates may be available for factor replacement in some countries.

The amino acid sequences of certain clotting (also known as “coagulation”) factors are provided below.

Fibrinogen (Clotting Factor I) alpha chain Amino Acid Sequence:
>sp|P02671|FIBA_HUMAN Fibrinogen alpha chain OS = Homo sapiens OX = 9606
GN = FGA PE = 1 SV = 2
SEQ ID NO: 1
MFSMRIVCLVLSVVGTAWTADSGEGDFLAEGGGVRGPRVVERHQSACKDSDWPFCS
DEDWNYKCPSGCRMKGLIDEVNQDFTNRINKLKNSLFEYQKNNKDSHSLTTNIMEIL
RGDFSSANNRDNTYNRVSEDLRSRIEVLKRKVIEKVQHIQLLQKNVRAQLVDMKRLE
VDIDIKIRSCRGSCSRALAREVDLKDYEDQQKQLEQVIAKDLLPSRDRQHLPLIKMKP
VPDLVPGNFKSQLQKVPPEWKALTDMPQMRMELERPGGNEITRGGSTSYGTGSETES
PRNPSSAGSWNSGSSGPGSTGNRNPGSSGTGGTATWKPGSSGPGSTGSWNSGSSGTG
STGNQNPGSPRPGSTGTWNPGSSERGSAGHWTSESSVSGSTGQWHSESGSFRPDSPGS
GNARPNNPDWGTFEEVSGNVSPGTRREYHTEKLVTSKGDKELRTGKEKVTSGSTTTT
RRSCSKTVTKTVIGPDGHKEVTKEVVTSEDGSDCPEAMDLGTLSGIGTLDGFRHRHP
DEAAFFDTASTGKTFPGFFSPMLGEFVSETESRGSESGIFTNTKESSSHHPGIAEFPSRG
KSSSYSKQFTSSTSYNRGDSTFESKSYKMADEAGSEADHEGTHSTKRGHAKSRPVRD
CDDVLQTHPSGTQSGIFNIKLPGSSKIFSVYCDQETSLGGWLLIQQRMDGSLNFNRTW
QDYKRGFGSLNDEGEGEFWLGNDYLHLLTQRGSVLRVELEDWAGNEAYAEYHFRV
GSEAEGYALQVSSYEGTAGDALIEGSVEEGAEYTSHNNMQFSTFDRDADQWEENCA
EVYGGGWWYNNCQAANLNGIYYPGGSYDPRNNSPYEIENGVVWVSFRGADYSLRA
VRMKIRPLVTQ
Fibrinogen (Clotting Factor I) beta chain Amino Acid Sequence:
>sp|P02675|FIBB_HUMAN Fibrinogen beta chain OS = Homo sapiens OX = 9606
GN = FGB PE = 1 SV = 2
SEQ ID NO: 2
MKRMVSWSFHKLKTMKHLLLLLLCVFLVKSQGVNDNEEGFFSARGHRPLDKKREEA
PSLRPAPPPISGGGYRARPAKAAATQKKVERKAPDAGGCLHADPDLGVLCPTGCQLQ
EALLQQERPIRNSVDELNNNVEAVSQTSSSSFQYMYLLKDLWQKRQKQVKDNENVV
NEYSSELEKHQLYIDETVNSNIPTNLRVLRSILENLRSKIQKLESDVSAQMEYCRTPCT
VSCNIPVVSGKECEEIIRKGGETSEMYLIQPDSSVKPYRVYCDMNTENGGWTVIQNRQ
DGSVDFGRKWDPYKQGFGNVATNTDGKNYCGLPGEYWLGNDKISQLTRMGPTELLI
EMEDWKGDKVKAHYGGFTVQNEANKYQISVNKYRGTAGNALMDGASQLMGENRT
MTIHNGMFFSTYDRDNDGWLTSDPRKQCSKEDGGGWWYNRCHAANPNGRYYWGG
QYTWDMAKHGTDDGVVWMNWKGSWYSMRKMSMKIRPFFPQQ
Fibrinogen (Clotting Factor I) gamma chain Amino Acid Sequence:
>sp|P02679|FIBG_HUMAN Fibrinogen gamma chain OS = Homo sapiens OX = 9606
GN = FGG PE = 1 SV = 3
SEQ ID NO: 3
MSWSLHPRNLILYFYALLFLSSTCVAYVATRDNCCILDERFGSYCPTTCGIADFLSTYQ
TKVDKDLQSLEDILHQVENKTSEVKQLIKAIQLTYNPDESSKPNMIDAATLKSRKMLE
EIMKYEASILTHDSSIRYLQEIYNSNNQKIVNLKEKVAQLEAQCQEPCKDTVQIHDITG
KDCQDIANKGAKQSGLYFIKPLKANQQFLVYCEIDGSGNGWTVFQKRLDGSVDFKK
NWIQYKEGFGHLSPTGTTEFWLGNEKIHLISTQSAIPYALRVELEDWNGRTSTADYA
MFKVGPEADKYRLTYAYFAGGDAGDAFDGFDFGDDPSDKFFTSHNGMQFSTWDND
NDKFEGNCAEQDGSGWWMNKCHAGHLNGVYYQGGTYSKASTPNGYDNGIIWATW
KTRWYSMKKTTMKIIPFNRLTIGEGQQHHLGGAKQVRPEHPAETEYDSLYPEDDL
Clotting Factor II (Protrombin) Amino Acid Sequence
>sp|P00734|THRB_HUMAN Prothrombin OS = Homo sapiens OX = 9606 GN = F2 PE = 1
SV = 2
SEQ ID NO: 4
MAHVRGLQLPGCLALAALCSLVHSQHVFLAPQQARSLLQRVRRANTFLEEVRKGNL
ERECVEETCSYEEAFEALESSTATDVFWAKYTACETARTPRDKLAACLEGNCAEGLG
TNYRGHVNITRSGIECQLWRSRYPHKPEINSTTHPGADLQENFCRNPDSSTTGPWCYT
TDPTVRRQECSIPVCGQDQVTVAMTPRSEGSSVNLSPPLEQCVPDRGQQYQGRLAVT
THGLPCLAWASAQAKALSKHQDENSAVQLVENFCRNPDGDEEGVWCYVAGKPGDF
GYCDLNYCEEAVEEETGDGLDEDSDRAIEGRTATSEYQTFFNPRTFGSGEADCGLRPL
FEKKSLEDKTERELLESYIDGRIVEGSDAEIGMSPWQVMLFRKSPQELLCGASLISDR
WVLTAAHCLLYPPWDKNFTENDLLVRIGKHSRTRYERNIEKISMLEKIYIHPRYNWRE
NLDRDIALMKLKKPVAFSDYIHPVCLPDRETAASLLQAGYKGRVTGWGNLKETWTA
NVGKGQPSVLQVVNLPIVERPVCKDSTRIRITDNMFCAGYKPDEGKRGDACEGDSGG
PFVMKSPFNNRWYQMGIVSWGEGCDRDGKYGFYTHVFRLKKWIQKVIDQFGE
Clotting Factor V Amino Acid Sequence
>sp|P12259|FA5_HUMAN Coagulation factor V OS = Homo sapiens OX = 9606 GN = F5
PE = 1 SV = 4
SEQ ID NO: 5
MFPGCPRLWVLVVLGTSWVGWGSQGTEAAQLRQFYVAAQGISWSYRPEPTNSSLNL
SVTSFKKIVYREYEPYFKKEKPQSTISGLLGPTLYAEVGDIIKVHFKNKADKPLSIHPQ
GIRYSKLSEGASYLDHTFPAEKMDDAVAPGREYTYEWSISEDSGPTHDDPPCLTHIYY
SHENLIEDFNSGLIGPLLICKKGTLTEGGTQKTFDKQIVLLFAVFDESKSWSQSSSLMY
TVNGYVNGTMPDITVCAHDHISWHLLGMSSGPELFSIHFNGQVLEQNHHKVSAITLV
SATSTTANMTVGPEGKWIISSLTPKHLQAGMQAYIDIKNCPKKTRNLKKITREQRRH
MKRWEYFIAAEEVIWDYAPVIPANMDKKYRSQHLDNFSNQIGKHYKKVMYTQYED
ESFTKHTVNPNMKEDGILGPIIRAQVRDTLKIVFKNMASRPYSIYPHGVTFSPYEDEVN
SSFTSGRNNTMIRAVQPGETYTYKWNILEFDEPTENDAQCLTRPYYSDVDIMRDIASG
LIGLLLICKSRSLDRRGIQRAADIEQQAVFAVFDENKSWYLEDNINKFCENPDEVKRD
DPKFYESNIMSTINGYVPESITTLGFCFDDTVQWHFCSVGTQNEILTIHFTGHSFIYGKR
HEDTLTLFPMRGESVTVTMDNVGTWMLTSMNSSPRSKKLRLKFRDVKCIPDDDEDS
YEIFEPPESTVMATRKMHDRLEPEDEESDADYDYQNRLAAALGIRSFRNSSLNQEEEE
FNLTALALENGTEFVSSNTDIIVGSNYSSPSNISKFTVNNLAEPQKAPSHQQATTAGSP
LRHLIGKNSVLNSSTAEHSSPYSEDPIEDPLQPDVTGIRLLSLGAGEFKSQEHAKHKGP
KVERDQAAKHRFSWMKLLAHKVGRHLSQDTGSPSGMRPWEDLPSQDTGSPSRMRP
WKDPPSDLLLLKQSNSSKILVGRWHLASEKGSYEIIQDTDEDTAVNNWLISPQNASRA
WGESTPLANKPGKQSGHPKFPRVRHKSLQVRQDGGKSRLKKSQFLIKTRKKKKEKH
THHAPLSPRTFHPLRSEAYNTFSERRLKHSLVLHKSNETSLPTDLNQTLPSMDFGWIAS
LPDHNQNSSNDTGQASCPPGLYQTVPPEEHYQTFPIQDPDQMHSTSDPSHRSSSPELSE
MLEYDRSHKSFPTDISQMSPSSEHEVWQTVISPDLSQVTLSPELSQTNLSPDLSHTTLSP
ELIQRNLSPALGQMPISPDLSHTTLSPDLSHTTLSLDLSQTNLSPELSQTNLSPALGQMP
LSPDLSHTTLSLDFSQTNLSPELSHMTLSPELSQTNLSPALGQMPISPDLSHTTLSLDES
QTNLSPELSQTNLSPALGQMPLSPDPSHTTLSLDLSQTNLSPELSQTNLSPDLSEMPLF
ADLSQIPLTPDLDQMTLSPDLGETDLSPNFGQMSLSPDLSQVTLSPDISDTTLLPDLSQI
SPPPDLDQIFYPSESSQSLLLQEFNESFPYPDLGQMPSPSSPTLNDTFLSKEFNPLVIVGL
SKDGTDYIEIIPKEEVQSSEDDYAEIDYVPYDDPYKTDVRTNINSSRDPDNIAAWYLRS
NNGNRRNYYIAAEEISWDYSEFVQRETDIEDSDDIPEDTTYKKVVFRKYLDSTFTKRD
PRGEYEEHLGILGPIIRAEVDDVIQVRFKNLASRPYSLHAHGLSYEKSSEGKTYEDDSP
EWFKEDNAVQPNSSYTYVWHATERSGPESPGSACRAWAYYSAVNPEKDIHSGLIGPL
LICQKGILHKDSNMPMDMREFVLLFMTFDEKKSWYYEKKSRSSWRLTSSEMKKSHE
FHAINGMIYSLPGLKMYEQEWVRLHLLNIGGSQDIHVVHFHGQTLLENGNKQHQLG
VWPLLPGSFKTLEMKASKPGWWLLNTEVGENQRAGMQTPFLIMDRDCRMPMGLST
GIISDSQIKASEFLGYWEPRLARLNNGGSYNAWSVEKLAAEFASKPWIQVDMQKEVII
TGIQTQGAKHYLKSCYTTEFYVAYSSNQINWQIFKGNSTRNVMYFNGNSDASTIKEN
QFDPPIVARYIRISPTRAYNRPTLRLELQGCEVNGCSTPLGMENGKIENKQITASSFKKS
WWGDYWEPFRARLNAQGRVNAWQAKANNNKQWLEIDLLKIKKITAIITQGCKSLSS
EMYVKSYTIHYSEQGVEWKPYRLKSSMVDKIFEGNTNTKGHVKNFFNPPIISRFIRVIP
KTWNQSIALRLELFGCDIY
Clotting Factor VII Amino Acid Sequence:
>sp|P08709|FA7_HUMAN Coagulation factor VII OS = Homo sapiens OX-9606 GN = F7
PE = 1 SV = 1
SEQ ID NO: 6
MVSQALRLLCLLLGLQGCLAAGGVAKASGGETRDMPWKPGPHRVFVTQEEAHGVL
HRRRRANAFLEELRPGSLERECKEEQCSFEEAREIFKDAERTKLFWISYSDGDQCASSP
CQNGGSCKDQLQSYICFCLPAFEGRNCETHKDDQLICVNENGGCEQYCSDHTGTKRS
CRCHEGYSLLADGVSCTPTVEYPCGKIPILEKRNASKPQGRIVGGKVCPKGECPWQVL
LLVNGAQLCGGTLINTIWVVSAAHCFDKIKNWRNLIAVLGEHDLSEHDGDEQSRRVA
QVIIPSTYVPGTTNHDIALLRLHQPVVLTDHVVPLCLPERTFSERTLAFVRFSLVSGWG
QLLDRGATALELMVLNVPRLMTQDCLQQSRKVGDSPNITEYMFCAGYSDGSKDSCK
GDSGGPHATHYRGTWYLTGIVSWGQGCATVGHFGVYTRVSQYIEWLQKLMRSEPRP
GVLLRAPFP
Clotting Factor VIII Amino Acid Sequence:
>sp|P00451|FA8_HUMAN Coagulation factor VIII OS = Homo sapiens OX = 9606
GN = F8 PE = 1 SV = 1
SEQ ID. NO: 7
MQIELSTCFFLCLLRFCFSATRRYYLGAVELSWDYMQSDLGELPVDARFPPRVPKSFP
FNTSVVYKKTLFVEFTDHLFNIAKPRPPWMGLLGPTIQAEVYDTVVITLKNMASHPVS
LHAVGVSYWKASEGAEYDDQTSQREKEDDKVFPGGSHTYVWQVLKENGPMASDPL
CLTYSYLSHVDLVKDLNSGLIGALLVCREGSLAKEKTQTLHKFILLFAVFDEGKSWHS
ETKNSLMQDRDAASARAWPKMHTVNGYVNRSLPGLIGCHRKSVYWHVIGMGTTPE
VHSIFLEGHTFLVRNHRQASLEISPITFLTAQTLLMDLGQFLLFCHISSHQHDGMEAYV
KVDSCPEEPQLRMKNNEEAEDYDDDLTDSEMDVVRFDDDNSPSFIQIRSVAKKHPKT
WVHYIAAEEEDWDYAPLVLAPDDRSYKSQYLNNGPQRIGRKYKKVRFMAYTDETF
KTREAIQHESGILGPLLYGEVGDTLLIIFKNQASRPYNIYPHGITDVRPLYSRRLPKGVK
HLKDFPILPGEIFKYKWTVTVEDGPTKSDPRCLTRYYSSFVNMERDLASGLIGPLLICY
KESVDQRGNQIMSDKRNVILFSVFDENRSWYLTENIQRFLPNPAGVQLEDPEFQASNI
MHSINGYVFDSLQLSVCLHEVAYWYILSIGAQTDFLSVFFSGYTFKHKMVYEDTLTLF
PFSGETVFMSMENPGLWILGCHNSDFRNRGMTALLKVSSCDKNTGDYYEDSYEDISA
YLLSKNNAIEPRSFSQNSRHPSTRQKQFNATTIPENDIEKTDPWFAHRTPMPKIQNVSS
SDLLMLLRQSPTPHGLSLSDLQEAKYETFSDDPSPGAIDSNNSLSEMTHFRPQLHHSG
DMVFTPESGLQLRLNEKLGTTAATELKKLDFKVSSTSNNLISTIPSDNLAAGTDNTSSL
GPPSMPVHYDSQLDTTLFGKKSSPLTESGGPLSLSEENNDSKLLESGLMNSQESSWGK
NVSSTESGRLFKGKRAHGPALLTKDNALFKVSISLLKTNKTSNNSATNRKTHIDGPSL
LIENSPSVWQNILESDTEFKKVTPLIHDRMLMDKNATALRLNHMSNKTTSSKNMEMV
QQKKEGPIPPDAQNPDMSFFKMLFLPESARWIQRTHGKNSLNSGQGPSPKQLVSLGPE
KSVEGQNFLSEKNKVVVGKGEFTKDVGLKEMVFPSSRNLFLTNLDNLHENNTHNQE
KKIQEEIEKKETLIQENVVLPQIHTVTGTKNFMKNLFLLSTRQNVEGSYDGAYAPVLQ
DFRSLNDSTNRTKKHTAHFSKKGEEENLEGLGNQTKQIVEKYACTTRISPNTSQQNFV
TQRSKRALKQFRLPLEETELEKRIIVDDTSTQWSKNMKHLTPSTLTQIDYNEKEKGAIT
QSPLSDCLTRSHSIPQANRSPLPIAKVSSFPSIRPIYLTRVLFQDNSSHLPAASYRKKDSG
VQESSHFLQGAKKNNLSLAILTLEMTGDQREVGSLGTSATNSVTYKKVENTVLPKPD
LPKTSGKVELLPKVHIYQKDLFPTETSNGSPGHLDLVEGSLLQGTEGAIKWNEANRP
GKVPFLRVATESSAKTPSKLLDPLAWDNHYGTQIPKEEWKSQEKSPEKTAFKKKDTIL
SLNACESNHAIAAINEGQNKPEIEVTWAKQGRTERLCSQNPPVLKRHQREITRTTLQS
DQEEIDYDDTISVEMKKEDFDIYDEDENQSPRSFQKKTRHYFIAAVERLWDYGMSSSP
HVLRNRAQSGSVPQFKKVVFQEFTDGSFTQPLYRGELNEHLGLLGPYIRAEVEDNIM
VTFRNQASRPYSFYSSLISYEEDQRQGAEPRKNFVKPNETKTYFWKVQHHMAPTKDE
FDCKAWAYFSDVDLEKDVHSGLIGPLLVCHTNTLNPAHGRQVTVQEFALFFTIFDET
KSWYFTENMERNCRAPCNIQMEDPTFKENYRFHAINGYIMDTLPGLVMAQDQRIRW
YLLSMGSNENIHSIHFSGHVFTVRKKEEYKMALYNLYPGVFETVEMLPSKAGIWRVE
CLIGEHLHAGMSTLFLVYSNKCQTPLGMASGHIRDFQITASGQYGQWAPKLARLHYS
GSINAWSTKEPFSWIKVDLLAPMIIHGIKTQGARQKFSSLYISQFIIMYSLDGKKWQTY
RGNSTGTLMVFFGNVDSSGIKHNIFNPPIIARYIRLHPTHYSIRSTLRMELMGCDLNSCS
MPLGMESKAISDAQITASSYFTNMFATWSPSKARLHLQGRSNAWRPQVNNPKEWLQ
VDFQKTMKVTGVTTQGVKSLLTSMYVKEFLISSSQDGHQWTLFFQNGKVKVFQGNQ
DSFTPVVNSLDPPLLTRYLRIHPQSWVHQIALRMEVLGCEAQDLY SEQ ID NO: 7
Clotting Factor IX Amino Acid Sequence
>sp|P00740|FA9_HUMAN Coagulation factor IX OS = Homo sapiens OX = 9606 GN = F9
PE = 1 SV = 2
SEQ ID NO: 8
MQRVNMIMAESPGLITICLLGYLLSAECTVFLDHENANKILNRPKRYNSGKLEEFVQG
NLERECMEEKCSFEEAREVFENTERTTEFWKQYVDGDQCESNPCLNGGSCKDDINSY
ECWCPFGFEGKNCELDVTCNIKNGRCEQFCKNSADNKVVCSCTEGYRLAENQKSCEP
AVPFPCGRVSVSQTSKLTRAETVFPDVDYVNSTEAETILDNITQSTQSENDFTRVVGG
EDAKPGQFPWQVVLNGKVDAFCGGSIVNEKWIVTAAHCVETGVKITVVAGEHNIEE
TEHTEQKRNVIRIIPHHNYNAAINKYNHDIALLELDEPLVLNSYVTPICIADKEYTNIFL
KFGSGYVSGWGRVFHKGRSALVLQYLRVPLVDRATCLRSTKFTIYNNMFCAGFHEG
GRDSCQGDSGGPHVTEVEGTSFLTGIISWGEECAMKGKYGIYTKVSRYVNWIKEKTK
LT
Clotting Factor X Amino Acid Sequence:
>sp|P00742|FA10_HUMAN Coagulation factor X OS = Homo sapiens OX = 9606
GN = F10 PE = 1 SV = 2
SEQ ID NO: 9
MGRPLHLVLLSASLAGLLLLGESLFIRREQANNILARVTRANSFLEEMKKGHLERECM
EETCSYEEAREVFEDSDKTNEFWNKYKDGDQCETSPCQNQGKCKDGLGEYTCTCLE
GFEGKNCELFTRKLCSLDNGDCDQFCHEEQNSVVCSCARGYTLADNGKACIPTGPYP
CGKQTLERRKRSVAQATSSSGEAPDSITWKPYDAADLDPTENPFDLLDFNQTQPERG
DNNLTRIVGGQECKDGECPWQALLINEENEGFCGGTILSEFYILTAAHCLYQAKRFKV
RVGDRNTEQEEGGEAVHEVEVVIKHNRFTKETYDFDIAVLRLKTPITFRMNVAPACL
PERDWAESTLMTQKTGIVSGFGRTHEKGRQSTRLKMLEVPYVDRNSCKLSSSFIITQN
MFCAGYDTKQEDACQGDSGGPHVTRFKDTYFVTGIVSWGEGCARKGKYGIYTKVT
AFLKWIDRSMKTRGLPKAKSHAPEVITSSPLK
Clotting Factor XI Amino Acid Sequence
>sp|P03951|FA11_HUMAN Coagulation factor XI OS = Homo sapiens OX = 9606
GN = F11 PE = 1 SV = 1
SEQ ID NO: 10
MIFLYQVVHFILFTSVSGECVTQLLKDTCFEGGDITTVFTPSAKYCQVVCTYHPRCLLF
TFTAESPSEDPTRWFTCVLKDSVTETLPRVNRTAAISGYSFKQCSHQISACNKDIYVDL
DMKGINYNSSVAKSAQECQERCTDDVHCHFFTYATRQFPSLEHRNICLLKHTQTGTP
TRITKLDKVVSGFSLKSCALSNLACIRDIFPNTVFADSNIDSVMAPDAFVCGRICTHHP
GCLFFTFFSQEWPKESQRNLCLLKTSESGLPSTRIKKSKALSGFSLQSCRHSIPVFCHSS
FYHDTDFLGEELDIVAAKSHEACQKLCTNAVRCQFFTYTPAQASCNEGKGKCYLKLS
SNGSPTKILHGRGGISGYTLRLCKMDNECTTKIKPRIVGGTASVRGEWPWQVTLHTTS
PTQRHLCGGSIIGNQWILTAAHCFYGVESPKILRVYSGILNQSEIKEDTSFFGVQEIIIHD
QYKMAESGYDIALTNEECQKRYRGHKITHKMICAGYREGGKDACKGDSGGPLSCKH
NEVWHLVGITSWGEGCAQRERPGVYTNVVEYVDWILEKTQAV
Clotting Factor XIII alpha chain Amino Acid Sequence:
>sp|P00488|F13A_HUMAN Coagulation factor XIII A chain OS = Homo sapiens
OX = 9606 GN = F13A1 PE = 1 SV = 4
SEQ ID NO: 11
MSETSRTAFGGRRAVPPNNSNAAEDDLPTVELQGVVPRGVNLQEFLNVTSVHLFKER
WDTNKVDHHTDKYENNKLIVRRGQSFYVQIDFSRPYDPRRDLFRVEYVIGRYPQENK
GTYIPVPIVSELQSGKWGAKIVMREDRSVRLSIQSSPKCIVGKFRMYVAVWTPYGVLR
TSRNPETDTYILFNPWCEDDAVYLDNEKEREEYVLNDIGVIFYGEVNDIKTRSWSYGQ
FEDGILDTCLYVMDRAQMDLSGRGNPIKVSRVGSAMVNAKDDEGVLVGSWDNIYA
YGVPPSAWTGSVDILLEYRSSENPVRYGQCWVFAGVENTFLRCLGIPARIVTNYFSAH
DNDANLQMDIFLEEDGNVNSKLTKDSVWNYHCWNEAWMTRPDLPVGFGGWQAVD
STPQENSDGMYRCGPASVQAIKHGHVCFQFDAPFVFAEVNSDLIYITAKKDGTHVVE
NVDATHIGKLIVTKQIGGDGMMDITDTYKFQEGQEEERLALETALMYGAKKPLNTEG
VMKSRSNVDMDFEVENAVLGKDFKLSITFRNNSHNRYTITAYLSANITFYTGVPKAEF
KKETFDVTLEPLSFKKEAVLIQAGEYMGQLLEQASLHFFVTARINETRDVLAKQKSTV
LTIPEIIIKVRGTQVVGSDMTVTVQFTNPLKETLRNVWVHLDGPGVTRPMKKMFREIR
PNSTVQWEEVCRPWVSGHRKLIASMSSDSLRHVYGELDVQIQRRPSM
Clotting Factor XIII beta chain Amino Acid Sequence:
>sp|P05160|F13B_HUMAN Coagulation factor XIII B chain OS = Homo sapiens
OX = 9606 GN = F13B PE = 1 SV = 3
SEQ ID NO: 12
MRLKNLTFIIILIISGELYAEEKPCGFPHVENGRIAQYYYTFKSFYFPMSIDKKLSFFCL
AGYTTESGRQEEQTTCTTEGWSPEPRCFKKCTKPDLSNGYISDVKLLYKIQENMRYG
CASGYKTTGGKDEEVVQCLSDGWSSQPTCRKEHETCLAPELYNGNYSTTQKTFKVK
DKVQYECATGYYTAGGKKTEEVECLTYGWSLTPKCTKLKCSSLRLIENGYFHPVKQT
YEEGDVVQFFCHENYYLSGSDLIQCYNFGWYPESPVCEGRRNRCPPPPLPINSKIQTHS
TTYRHGEIVHIECELNFEIHGSAEIRCEDGKWTEPPKCIEGQEKVACEEPPFIENGAANL
HSKIYYNGDKVTYACKSGYLLHGSNEITCNRGKWTLPPECVENNENCKHPPVVMNG
AVADGILASYATGSSVEYRCNEYYLLRGSKISRCEQGKWSSPPVCLEPCTVNVDYMN
RNNIEMKWKYEGKVLHGDLIDFVCKQGYDLSPLTPLSELSVQCNRGEVKYPLCTRKE
SKGMCTSPPLIKHGVIISSTVDTYENGSSVEYRCFDHHFLEGSREAYCLDGMWTTPPL
CLEPCTLSFTEMEKNNLLLKWDFDNRPHILHGEYIEFICRGDTYPAELYITGSILRMQC
DRGQLKYPRCIPRQSTLSYQEPLRT
Von Willebrand Factor Amino Acid Sequence:
>sp|P04275|VWF_HUMAN von Willebrand factor OS = Homo sapiens OX = 9606
GN = VWF PE = 1 SV = 4
SEQ ID NO: 13
MIPARFAGVLLALALILPGTLCAEGTRGRSSTARCSLFGSDFVNTFDGSMYSFAGYCS
YLLAGGCQKRSFSIIGDFQNGKRVSLSVYLGEFFDIHLFVNGTVTQGDQRVSMPYASK
GLYLETEAGYYKLSGEAYGFVARIDGSGNFQVLLSDRYFNKTCGLCGNFNIFAEDDF
MTQEGTLTSDPYDFANSWALSSGEQWCERASPPSSSCNISSGEMQKGLWEQCQLLKS
TSVFARCHPLVDPEPFVALCEKTLCECAGGLECACPALLEYARTCAQEGMVLYGWT
DHSACSPVCPAGMEYRQCVSPCARTCQSLHINEMCQERCVDGCSCPEGQLLDEGLCV
ESTECPCVHSGKRYPPGTSLSRDCNTCICRNSQWICSNEECPGECLVTGQSHFKSFDNR
YFTFSGICQYLLARDCQDHSFSIVIETVQCADDRDAVCTRSVTVRLPGLHNSLVKLKH
GAGVAMDGQDVQLPLLKGDLRIQHTVTASVRLSYGEDLQMDWDGRGRLLVKLSPV
YAGKTCGLCGNYNGNQGDDFLTPSGLAEPRVEDFGNAWKLHGDCQDLQKQHSDPC
ALNPRMTRFSEEACAVLTSPTFEACHRAVSPLPYLRNCRYDVCSCSDGRECLCGALA
SYAAACAGRGVRVAWREPGRCELNCPKGQVYLQCGTPCNLTCRSLSYPDEECNEAC
LEGCFCPPGLYMDERGDCVPKAQCPCYYDGEIFQPEDIFSDHHTMCYCEDGFMHCT
MSGVPGSLLPDAVLSSPLSHRSKRSLSCRPPMVKLVCPADNLRAEGLECTKTCQNYD
LECMSMGCVSGCLCPPGMVRHENRCVALERCPCFHQGKEYAPGETVKIGCNTCVCQ
DRKWNCTDHVCDATCSTIGMAHYLTFDGLKYLFPGECQYVLVQDYCGSNPGTFRIL
VGNKGCSHPSVKCKKRVTILVEGGEIELFDGEVNVKRPMKDETHFEVVESGRYIILLL
GKALSVVWDRHLSISVVLKQTYQEKVCGLCGNFDGIQNNDLTSSNLQVEEDPVDFG
NSWKVSSQCADTRKVPLDSSPATCHNNIMKQTMVDSSCRILTSDVFQDCNKLVDPEP
YLDVCIYDTCSCESIGDCACFCDTIAAYAHVCAQHGKVVTWRTATLCPQSCEERNLR
ENGYECEWRYNSCAPACQVTCQHPEPLACPVQCVEGCHAHCPPGKILDELLQTCVDP
EDCPVCEVAGRRFASGKKVTLNPSDPEHCQICHCDVVNLTCEACQEPGGLVVPPTDA
PVSPTTLYVEDISEPPLHDFYCSRLLDLVFLLDGSSRLSEAEFEVLKAFVVDMMERLRI
SQKWVRVAVVEYHDGSHAYIGLKDRKRPSELRRIASQVKYAGSQVASTSEVLKYTLF
QIFSKIDRPEASRITLLLMASQEPQRMSRNFVRYVQGLKKKKVIVIPVGIGPHANLKQI
RLIEKQAPENKAFVLSSVDELEQQRDEIVSYLCDLAPEAPPPTLPPDMAQVTVGPGLL
GVSTLGPKRNSMVLDVAFVLEGSDKIGEADFNRSKEFMEEVIQRMDVGQDSIHVTVL
QYSYMVTVEYPFSEAQSKGDILQRVREIRYQGGNRTNTGLALRYLSDHSFLVSQGDR
EQAPNLVYMVTGNPASDEIKRLPGDIQVVPIGVGPNANVQELERIGWPNAPILIQDFET
LPREAPDLVLQRCCSGEGLQIPTLSPAPDCSQPLDVILLLDGSSSFPASYFDEMKSFAK
AFISKANIGPRLTQVSVLQYGSITTIDVPWNVVPEKAHLLSLVDVMQREGGPSQIGDA
LGFAVRYLTSEMHGARPGASKAVVILVTDVSVDSVDAAADAARSNRVTVFPIGIGDR
YDAAQLRILAGPAGDSNVVKLQRIEDLPTMVTLGNSFLHKLCSGFVRICMDEDGNEK
RPGDVWTLPDQCHTVTCQPDGQTLLKSHRVNCDRGLRPSCPNSQSPVKVEETCGCR
WTCPCVCTGSSTRHIVTFDGQNFKLTGSCSYVLFQNKEQDLEVILHNGACSPGARQG
CMKSIEVKHSALSVELHSDMEVTVNGRLVSVPYVGGNMEVNVYGAIMHEVRENHL
GHIFTFTPQNNEFQLQLSPKTFASKTYGLCGICDENGANDFMLRDGTVTTDWKTLVQ
EWTVQRPGQTCQPILEEQCLVPDSSHCQVLLLPLFAECHKVLAPATFYAICQQDSCHQ
EQVCEVIASYAHLCRTNGVCVDWRTPDFCAMSCPPSLVYNHCEHGCPRHCDGNVSS
CGDHPSEGCFCPPDKVMLEGSCVPEEACTQCIGEDGVQHQFLEAWVPDHQPCQICTC
LSGRKVNCTTQPCPTAKAPTCGLCEVARLRQNADQCCPEYECVCDPVSCDLPPVPHC
ERGLQPTLTNPGECRPNFTCACRKEECKRVSPPSCPPHRLPTLRKTQCCDEYECACNC
VNSTVSCPLGYLASTATNDCGCTTTTCLPDKVCVHRSTIYPVGQFWEEGCDVCTCTD
MEDAVMGLRVAQCSQKPCEDSCRSGFTYVLHEGECCGRCLPSACEVVTGSPRGDSQ
SSWKSVGSQWASPENPCLINECVRVKEEVFIQQRNVSCPQLEVPVCPSGFQLSCKTSA
CCPSCRCERMEACMLNGTVIGPGKTVMIDVCTTCRCMVQVGVISGFKLECRKTTCNP
CPLGYKEENNTGECCGRCLPTACTIQLRGGQIMTLKRDETLQDGCDTHFCKVNERGE
YFWEKRVTGCPPFDEHKCLAEGGKIMKIPGTCCDTCEEPECNDITARLQYVKVGSCK
SEVEVDIHYCQGKCASKAMYSIDINDVQDQCSCCSPTRTEPMQVALHCTNGSVVYHE
VLNAMECKCSPRKCSK
Antithrombin III Amino Acid Sequence:
>sp|P01008|ANT3_HUMAN Antithrombin-III OS = Homo sapiens OX = 9606
GN = SERPINC1 PE = 1 SV = 1
SEQ ID NO: 14
MYSNVIGTVTSGKRKVYLLSLLLIGFWDCVTCHGSPVDICTAKPRDIPMNPMCIYRSP
EKKATEDEGSEQKIPEATNRRVWELSKANSRFATTFYQHLADSKNDNDNIFLSPLSIST
AFAMTKLGACNDTLQQLMEVFKFDTISEKTSDQIHFFFAKLNCRLYRKANKSSKLVS
ANRLFGDKSLTFNETYQDISELVYGAKLQPLDFKENAEQSRAAINKWVSNKTEGRIT
DVIPSEAINELTVLVLVNTIYFKGLWKSKFSPENTRKELFYKADGESCSASMMYQEGK
FRYRRVAEGTQVLELPFKGDDITMVLILPKPEKSLAKVEKELTPEVLQEWLDELEEM
MLVVHMPRFRIEDGFSLKEQLQDMGLVDLFSPEKSKLPGIVAEGRDDLYVSDAFHKA
FLEVNEEGSEAAASTAVVIAGRSLNPNRVTFKANRPFLVFIREVPLNTIIFMGRVANPC
VK

### Diagnosis of B Cell Resistance to Clotting Factor Treatment in Haemophilia

Diagnosis of haemophilia may involve complete blood count (CBC) to determine hemoglobin and platelet count is normal or low. Activated partial thromboplastin time (APTT) test can be done to measure bleeding time. Prothrombin time (PT) test can be done to determine if prothrombin time is normal.

Additionally, measurement of clotting factors can be done to determine whether these are low. Generally, if one or more of these tests results in the concentration of a clotting factor is below normal, a diagnosis of hemophilia is made. Normal physiological values and doses for treating haemophilia with B cell-mediated resistance for Factors I, II, V, VII, VIII, IX, X, XI, XIII, von Willebrand factor (VWF), and anti-thrombin III in a typical patient having a total blood volume of 5-6 liters are shown in Table 1 below. It is understood that the doses of exogenous clotting factors can be adjusted based on the patient's blood volume.

TABLE 1
Plasma Values for Clotting Factors in Haemophilia and Therapeutic Target Doses

| Clotting | | | | Therapeutic | | Dosage |
|---|---|---|---|---|---|---|
| Factor | Normal Range | | Haemophilia Range | Target | | per person |
| I | 20-40 | mg/ml | Severe: <2 mg/ml | 20-40 | g/ml | 100-240 g |
| | | | Moderate: 2-8 mg/ml | | | |
| | | | Mild | | | |
| II | 50-150 | μg/ml | 2%-50% of normal | 50-150 | mg/ml | 250-900 g |
| V | 4-14 | μg/ml | <50% of normal | 4-14 | mg/ml | 20 g-84 g |
| VII | .5 | μg/ml | Severe: <10% of normal | .5 | mg/ml | 2.5 g-3 g |
| | | | activity | | | |
| | | | Moderate: 10-20% of normal | | | |
| | | | activity | | | |
| | | | Mild: >20% of normal activity | | | |
| VIII (MW: | 100-200 | ng/ml | Severe: 0%-1% of normal | 100-200 | μg/ml | 500 mg-1.2 g |
| 330 kDa) | | | Moderate: 1%-5% of normal | | | |
| | | | Mild: 5%-40% of normal | | | |
| IX (MW: | 3-6 | μg/ml | Severe: 0%-1% of normal | 3-6 | mg/ml | 15 g-36 g |
| 46.5482 kDa) | | | Moderate: 1%-5% of normal | | | |
| | | | Mild: 5%-40% of normal | | | |
| X | 6-8 | μg/ml | Severe: <1% of normal activity | 6-8 | mg/ml | 30 g-48 g |
| | | | Moderate: 1-5% of normal | | | |
| | | | activity | | | |
| | | | Mild: 6-10% of normal activity | | | |
| XI (MW: | 3-7 | μg/ml | <5% of normal | 3-7 | mg/ml | 15 g-42 g |
| 160 kDa) | | | | | | |
| XIII | 14-28 | μg/ml | <20% | 14-28 | mg/ml | 70 g-168 g |
| VWF | 10 | μg/ml | <50% of normal | 10 | mg/ml | 50 g-60 g |
| Antithrombin | 150 | μg/ml | <80% activity of normal | 150 | mg/ml | 750 g-900 g |

III

Standard methods of detecting clotting factors involve measurements made using serum or blood of a patient. Detection of development of resistance to treatment due to the patients' antibody response to the injected coagulation factor is delayed and based on the clinical observation that the reconstitution dose needs to be increased.

Detection of B Cell Mediated Resistance to Clotting Factor Replacement in Patients with Haemophilia

We developed a new sensitive assay for detecting resistance to substitution factor therapy in patients with haemophilia involving detecting B cells producing antibodies in vitro directed toward clotting factors, including Factor I, Factor II, Factor V, Factor VII, Factor VIII, Factor IX, Factor X, Factor XI, Factor XIII, von Willebrand Factor (VWF), and antithrombin III.

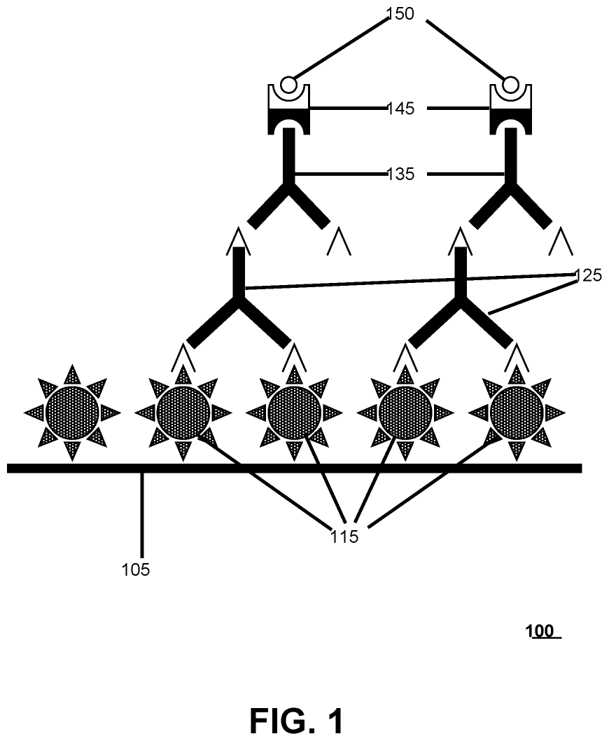

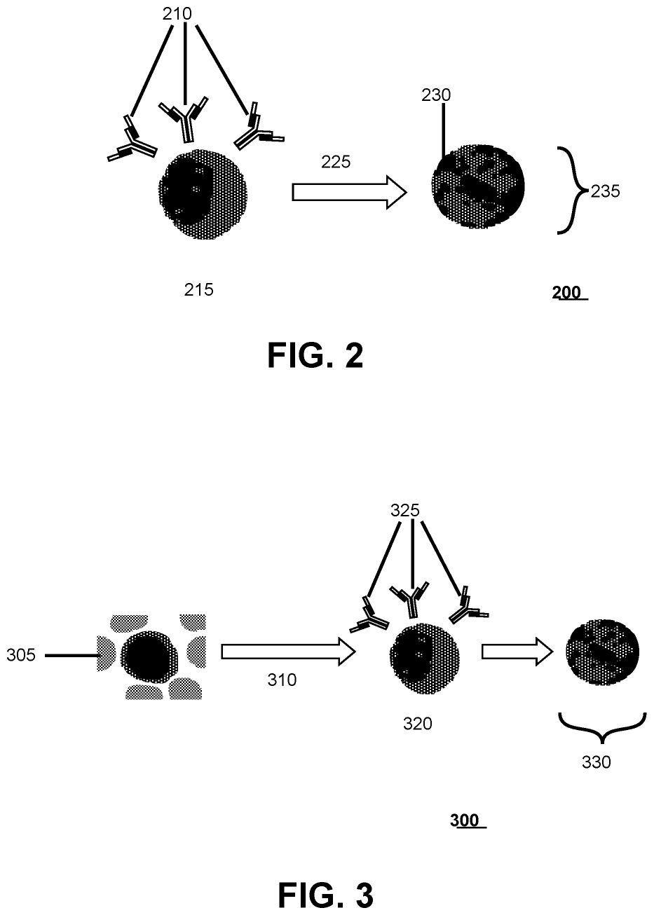

Improved assays include either a “direct B cell test,” and “indirect B cell test,” or both tests. The basic methods involve providing an assay plate (e.g., a well of a multiwell plate), attaching antigen (clotting factor) onto the plate, adding cell culture medium to the plate along with a sample of peripheral blood mononuclear cells (PBMCs). If B cells are present in the PBMC that produce antibodies against a clotting factor, the antibodies will bind around each ASC to the antigen on the plate. Then, the B cell-produced antibodies (e.g., immunoglobulin, Ig) can be visualized using an anti-human Ig detection antibody coupled to an enzyme. Subsequently, the precipitating substrate for the enzyme is added resulting in the appearance of a visible “spot” on the plate. Alternatively, a fluorescent label can be attached to the detection Ig that produces a fluorescent spot on the plate. FIGS. 1 and 2 depict the above described direct detection of clotting factor specific ASC. FIG. 3 depicts the “indirect B cell test” described in the following.

For an “indirect B cell test,” instead of placing freshly isolated PBMC direct ex vivo (without any additional stimulation) in the ELISPOT assay, the PBMC are first exposed to a B cell polyclonal activator, including but not limited to Interleukin 2 (IL2), and R848. Exposure of PBMC to a polyclonal activator stimulates B cell growth and induces B cells to produce antibodies. The indirect B cell test therefore enables the detection of resting B cells that do not produce antibody spontaneously. FIG. 3 depicts an indirect B cell test useful for this disclosure.

### Therapies for Haemophilia

Based on the information in Table 1, patients can be treated for different forms of B cell resistance to haemophilia by increasing the amount of the deficient clotting factor by about 1000-fold.

The primary strategy for treating resistance to substitution therapy in haemophilia is to reduce the numbers of clotting factor-specific B cells. Reduction can be accomplished by the administration of an intravenous dose of the clotting factor sufficient to produce about a 1,000-fold increase in in vivo concentration of the factor(s) compared to the normal physiological levels, followed by daily injections of the same factor in doses suited to maintain this high in vivo concentration for a duration of about 2 weeks. This regimen should suffice to trigger B-cell tolerance, i.e., to get rid of the existing clotting factor-specific B-cells. Use of high doses of replacement clotting factor may cause the desired antigen-specific B cell tolerance. As an alternative, injecting a clotting factor coupled with an immunotoxin (e.g., ricin) or immune suppressive agent should be suited to selectively kill or inactivate antigen-specific B cells. Ideally, the above therapies follow a general B cell-depleting intervention, such as rituximab injection. For a patient having Factor I deficiency, reduction of resistance to replacement (or substitution) therapy can be accomplished by intravenous injection of Factor I at a dose of about 100 g-about 240 g, depending on the patient's blood volume following rituximab injection. One can use the alpha chain (SEQ ID NO:1), the beta chain (SEQ ID NO: 2), or the gamma chain (SEQ ID NO:3).

It can be helpful to infuse clotting factors after injection of rituximab-arrx (“rituximab”), for example, sold under the trademark RIABNI™. The dose of rituximab can begin at a dose of about 100 mg/hr. In the absence of infusion toxicity, the dose can be increased at 30 minute intervals to a maximum dose of about 400 mg/hr.

For a patient having Factor II deficiency, reduction of resistance to replacement (or substitution) therapy can be accomplished by injecting Factor II (SEQ ID NO:4) at a dose of about 250 g-about 900 g, depending on the patient's total blood volume following rituximab injection.

For a patient having Factor V deficiency, reduction of resistance to replacement (or substitution) therapy can be accomplished by intravenous injection of Factor V (SEQ ID NO:5) at a dose of about 20 g-about 40 g, depending on the patient's blood volume following rituximab injection.

For a patient having Factor VII deficiency, reduction of resistance to replacement (or substitution) therapy is accomplished by injecting Factor VII (SEQ ID NO:6) at a dose of about 2.5 g-about 3.0 g, depending on the patient's total blood volume following rituximab injection.

Haemophilia A is characterized by a congenital deficiency in Factor VIII. To treat patients using substitution therapy, a clotting factor can be administered. Clotting factors may be conjugated with polyethylene glycol (PEG) to improve bioavailability, and recombinant Factor VIII is sold under the tradename JIVI®, a trademark of Bayer Healthcare. Factor VIII can be used to control bleeding episodes, as a perioperative management of bleeding during surgery, as a prophylactic to reduce frequency of bleeding episodes. Reduction of resistance to Factor VIII treatment can be accomplished by intravenous injection of Factor VIII (SEQ ID NO: 7) at a dose of about 500 mg to about 1.2 g, depending on the patient's blood volume following rituximab injection.

Haemophilia B is characterized by a congenital defect in Factor IX. To treat such patients, recombinant Factor IX (SEQ ID NO:8) can administered as, for example, FcFusion Protein sold under the tradename ALPROLIX®, a trademark of Genentech, Inc. Reduction of resistance to Factor IX replacement treatment can be accomplished by intravenous injection of Factor IX at a dose of about 15 g-36 g, depending on the patient's blood volume following rituximab injection.

For a patient having Factor X deficiency, reduction of resistance to Factor X replacement (or substitution) therapy can be accomplished by intravenous injection of Factor X (SEQ ID NO: 9) at a dose of about 30 g-about 48 g, depending on the patient's blood volume, following rituximab injection.

For patients having Factor XI deficiency, abrogation reduction of resistance to replacement (or substitution) therapy using recombinant Factor XI can be used by injecting Factor XI at a dose of about 15 g-about 42 g, depending on the patient's blood volume following rituximab injection.

For patients having Factor XIII deficiency, reduction of resistance to replacement (or substitution) therapy using recombinant Factor XIII can be used by intravenous injection of Factor XIII alpha chain (SEQ ID NO:10) or Factor XIII beta chain (SEQ ID NO:12) at a dose of about 70 g-about 168 g, depending on the patient's blood volume following rituximab injection.

For patients having von Willebrand (VWF) deficiency, abrogation reduction of resistance to replacement (or substitution) therapy using VWF can be used by intravenous injection of VWF (SEQ ID NO:13) at a dose of about 50 g-about 60 g. depending on the patient's blood volume following rituximab injection.

For patients having Antithrombin III deficiency, abrogation reduction of resistance to replacement therapy using recombinant Antithrombin III can be used by intravenous injection of Antithrombin III (SEQ ID NO:14) at a dose of about 750 g-about 900 g, depending on the patient's blood volume following rituximab injection.

### Aspects of this Disclosure

The following aspects are to be considered either individually, or along with other aspects.

One aspect is a method for detecting B-cell antibody response specific to a clotting factor in a patient having haemophilia and having B-cell-mediated resistance to substitution therapy using a direct B cell test.

A further aspect is a method for detecting a B-cell antibody response specific to a clotting in a patient having haemophilia (also known as hemophilia) and having B cell mediated resistance to substitution therapy using a direct B-cell test, comprising:
a) providing a sample of peripheral blood mononuclear cells (PBMCs) from said patient;
b) providing a cell culture plate having a clotting antigen attached thereto;
c) placing said sample of PBMCs in cell culture medium into said cell culture plate;
d) permitting B cells in said sample of PBMCs to secrete antibodies to said antigen; and
e) detecting binding of said antibodies to said antigen.

Another aspect is a method for detecting a B-cell antibody response to a clotting factor in a patient having haemophilia indicating the development of B cell mediated resistance to substitution therapy using an indirect B cell test, comprising:
a) adding a B-cell polyclonal activator to freshly isolated PBMCs for 3-8 days, thereby producing pre-stimulated PBMCs;
b) providing a cell culture plate having a clotting factor antigen attached thereto;
c) placing a sample of polyclonally pre-stimulated PBMCs from said patient in cell culture medium into said cell culture plate of step b);
d) permitting B cells in said sample of PBMCs to produce antibodies and said antibodies bind to antigen of step b); and
e) detecting binding of said antibodies to said antigen.

An additional aspect is one of the preceding or following aspects in which a B cell test is accomplished using ELISPOT or FluoroSpot™ methods.

A further aspect is a method for detecting a B-cell antibody response to a clotting factor in a patient having haemophilia and having B cell mediated resistance to substitution therapy using a direct ELISA test, comprising:
a) providing a sample of peripheral blood mononuclear cells (PBMCs) from said patient;
b) culturing these PBMCs with cell culture medium alone for ˜1 day; and
c) testing supernatant by ELISA for Factor VIII- or IX- or XI-specific antibodies.

A further aspect is any of the preceding or following aspects, wherein the clotting factor is Factor I, Factor II, Factor V, Factor VII, Factor VIII, Factor IX, Factor X, Factor XI, Factor XIII, von Willebrand Factor (VWF), and antithrombin III.

An additional aspect is a method for detecting a B-cell antibody response to a clotting factor in a patient having haemophilia and having B cell mediated resistance to substitution therapy using an indirect ELISA test, comprising:
a) providing a sample of peripheral blood mononuclear cells (PBMCs) from said patient;
b) culturing these PBMCs with a polyclonal B cell stimulator for 3-8 days; and
c) testing supernatant by ELISA for clotting factor-specific antibodies.

Another aspect is a method for treating a patient having haemophilia having developed resistance to reconstitution therapy and having clotting factor-specific B cells in vivo comprising:
a) infusing rituximab at a dose of 100 mg/hr, and if no toxicity is observed, increasing the dose every 30 minutes to a maximum dose of about 400 mg/hr.
b) injecting intravenously, a dose of clotting factor sufficient to produce about a 1,000-fold increase in in vivo concentration of the factor relative to its normal physiological concentration;
c) followed by daily injections of the same factor in a dose sufficient to maintain the high in vivo concentration for a duration of about 2 weeks.

An additional aspect is a method of any prior or following aspect, including optionally pre-treating a patient with rituximab to deplete B cells.

A further aspect is a method of a prior aspect, where said injections comprises injecting a clotting factor coupled with an immunotoxin (e.g., ricin) or immune suppressive agent to selectively kill or inactivate clotting factor-specific B cells.

Yet another aspect is a method of any prior or following aspect, further comprising assaying the success of the in vivo inactivation of clotting factor-specific B cells in said patient using an indirect B cell test.

A further aspect is a method of one or more prior aspects, wherein if the patient's haemophilia A does not improve, inject one of the therapeutic agents selected from the group consisting of Helixate® FS, Recombinate®, Kogenate® FS, Advate®, ReFacto®, Eloctate®, Nuwiq®, Adynovate®, Kovaltry®, Jivi®, and Xyntha®. Human plasma-derived preparations include Monarc-M®, Monoclate-P®, Hemofil M®, and Koate-DVI®.

An additional aspect is a method of one or more aspects, wherein if the patient's resistance to hemophilia A substitution therapy does not improve, inject one or more clotting factors selected from the group consisting of Factor VII, Factor IIa, Factor VIIa, Factor IXa, and Factor Xa.

Another aspect is a method of one or more aspects, wherein if the patient's hemophilia B does not improve with injection of Factor IX, inject one of the therapeutic agents selected from the group consisting of BeneFIX®, Rixubis®, Ixinity®, Alprolix®, Idelvion®, and Rebinyn®.

A further aspect is a method for detecting a B-cell antibody response to a clotting factor in a patient having haemophilia indicating the development of B cell mediated resistance to substitution therapy using a direct B cell test, comprising:
a) providing a sample of peripheral blood mononuclear cells (PBMCs) from said patient;
b) providing a cell culture plate having a membrane with clotting factor antigen attached thereto;
c) placing said sample of PBMCs in cell culture medium into said cell culture plate;
d) permitting B cells in said sample of PBMC to secrete antibodies to said antigen; and
e) detecting binding of said antibodies to said antigen.

Another aspect is a method for detecting a B-cell antibody response to a clotting factor in a patient having haemophilia indicating the development of B cell mediated resistance to substitution therapy using an indirect B cell test, comprising:
a) adding a B-cell polyclonal activator to freshly isolated PBMCs for 3-8 days
b) providing a cell culture plate having a clotting factor antigen attached thereto;
c) placing a sample of polyclonally pre-stimulated PBMCs from said patient in cell culture medium into said cell culture plate of step b);
d) permitting B cells in said sample of PBMCs to produce antibodies and said antibodies bind to antigen of step c); and
e) detecting binding of said antibodies to said antigen.

A further aspect is a method for detecting a B-cell antibody response to a clotting factor in a patient having haemophilia indicating the development of B cell mediated resistance to substitution therapy using a direct ELISA test, comprising:
a) providing a sample of peripheral blood mononuclear cells (PBMCs) from said patient;
b) culturing these PBMC with cell culture medium alone for ˜1 day; and
c) testing supernatant by ELISA for clotting factor-specific antibodies.

An additional aspect is a method for detecting a B-cell antibody response to a clotting factor in a patient having haemophilia with development of B cell mediated resistance to substitution therapy using an indirect ELISA test, comprising:
a) providing a sample of peripheral blood mononuclear cells (PBMCs) from said patient;
b) culturing these PBMCs with a polyclonal B cell activator for 3-8 days; and
c) testing supernatant by ELISA for clotting factor-specific antibodies.

A still further aspect is a method for treating a patient having haemophilia and having resistance to replacement or substitution therapy caused by clotting factor-specific B cells in vivo comprising:
a) injection of rituximab antibody for general B cell depletion; then
b) injecting intravenously, a dose of said clotting factor sufficient to produce about a 1,000-fold increase in in vivo concentration compared to the normal physiological concentration of the factor;
c) followed by daily injections of the same factor in a dose sufficient to maintain the high in vivo concentration for a duration of about 2 weeks.

Additionally, another aspect is a method of any preceding or following aspect, where said injecting comprises injecting a clotting factor coupled with an immunotoxin (e.g., ricin) or immune suppressive agent to selectively kill or inactivate clotting factor-specific B cells. Another aspect is a method of any preceding or following aspect, further comprising assaying said patient's progress using an indirect B cell test.

Another aspect is a method of any preceding or following aspects, wherein if the patient's resistance to substitution therapy for haemophilia A does not improve, inject one of the therapeutic agents selected from the group consisting of Helixate® FS, Recombinate®, Kogenate® FS, Advate®, ReFacto®, Eloctate®, Nuwiq®, Adynovate®, Kovaltry®, Jivi®, and Xyntha®. Human plasma-derived preparations include Monarc-M®, Monoclate-P®, Hemofil M®, and Koate-DVI®.

A still further aspect is a method of any preceding or following aspect, wherein if the patient's resistance to substitution therapy for haemophilia A does not improve, injecting one or more of Factor VII, or Factor IIa, or Factor VIIa, or Factor IXa, or Factor Xa.

Another aspect is a method of any preceding or following aspect, wherein if the patient's resistance to substitution therapy for treatment of haemophilia B does not improve with injection of Factor IX, injecting one of the therapeutic agents selected from the group consisting of BeneFIX®, Rixubis®, Ixinity®, Alprolix®, Idelvion®, and Rebinyn®.

Another aspect is a method of any preceding or following aspect, wherein said clotting factor is Factor I, or Factor II, or Factor V, or Factor VII, or Factor VIII, or Factor IX, or Factor X, or Factor XI, or Factor XIII, or von Willebrand Factor (VWF), or antithrombin III.

Another aspect is a method of any preceding or following aspect, wherein detection of antibodies to clotting factors as accomplished using ELISPOT, ELISA, or FluoroSpot™ methods.

### Specific Embodiments

Below are descriptions of materials and methods for detecting B cell antibody responses to clotting factors.

### Materials

### B-Cell ELISPOT

1) MultiScreenHTS® Filter Plates for ELISPOT (Millipore, Billerica, MA Cat. No. MSIPS4W).

2) Recombinant Factors I, II, V, VI, VII, VIII, IX, X, XI, XIII, VWF, and antithrombin III.

3) Recombinant human IL-2 for polyclonal B cell activation: Synonyms: T-cell growth factor (TCGF), interleukin-2. IL-2 is a powerful immunoregulatory lymphokine produced by T-cells in response to antigenic or mitogenic stimulation. IL-2 IL-2R signaling is required for T-cell proliferation and other fundamental functions which are essential for the immune response. IL-2 stimulates growth and differentiation of B-cells, NK cells, lymphokine activated killer cells, monocytes, macrophages and oligodendrocytes. (Peprotech, Rocky Hill, NJ; Cat. No. 200-02).

4) R848 for polyclonal activation: R848 is an imidazoquinoline compound with potent anti-viral activity. This low molecular weight synthetic molecule activates immune cells, including B cells via the TLR7 or TLR8 MyD88-dependent signaling pathway. Recently, R848 was shown to trigger NF-κB activation in cells expressing murine TLR8 when combined with poly(dT) (Enzo Life Sciences, Farmingdale, NY; Cat. No. ALX-420-038).

5) 2-mercaptoethanol for polyclonal activation: (Sigma-Aldrich, St. Louis, MO; Cat. No. M7154)

6) Detection antibodies for B-cell ELISPOT-Anti-Human IgG Fc PAN (1,2,3,4), biotin-conjugated (Hybridoma Reagent Laboratory, Baltimore, MD; Cat. No. HP6043B)

7) Detection antibodies for B-cell ELISPOT-Anti-Human IgM, biotin-conjugated (Hybridoma Reagent Laboratory, Baltimore, MDI Cat. No. HP6083B).

8) Streptavidin-AP (Alkaline Phosphatase) (Sigma-Aldrich, St. Louis, MO; Cat. No. S2890).

9) Vector Blue Alkaline Phosphatase Substrate Kit; (Vector Laboratories, Burlingame, CA; Cat. No. SK-5300).

10) RPMI Media 1640 for cell culture with L-Glutamine: (Invitrogen, Grand Island, NY; Cat. No. 11875).

11) Fetal bovine serum (FBS): (Invitrogen, Grand Island, NY; Cat. No. 16140).

### Antibody ELISA

1) Immuno 96 MicroWell™ Solid Plates: (Thermo Scientific, Nunc, Rochester, NY; Cat. No. 456537).

2) Detection antibodies for B-cell ELISPOT-Anti-Human IgG Fc PAN (1,2,3,4), biotin-conjugated: (Hybridoma Reagent Laboratory, Baltimore, MD; Cat. No. HP6043B).

3) Fetal bovine serum (FBS): (Invitrogen, Grand Island, NY; Cat. No. 16140).

4) Streptavidin-HRP (Horseradish Peroxidase): (BD Pharmingen, San Jose, CA; Cat. No. 557630).

5) TMB (3,3′,5,5′-Tetramethylbenzidine) substrate: (eBioscience, San Diego, CA; Cat. No. 00-4201-56).

### Direct B-Cell Test

Plasmablasts (also known as plasma cells, or effector B-cells, or antibody secreting cells ASC) are characterized by the production of antibodies. If ASC are specific for clotting factors they will produce antibodies against their specific antigen, which can be directly measured in vitro. Measurement of such antibodies can be detected using methods developed by Cellular Technology Ltd. (ImmunoSpot® is a trademark of Cellular Technology Ltd, Shaker Heights OH). FIGS. 1 and 2 depict the schematic strategy for carrying out a direct B cell test.

### Approach

Filter membranes are coated with clotting factor antigen(s). PBMCs are isolated from the blood, e.g., by density gradient centrifugation and transferred to the plates as described in U.S. Pat. No. 7,598,093. After suitable incubation time, allowing sufficient secretion and binding of antibodies to the coated antigen, the production of clotting factor-specific antibodies is detected by either fluorescent or enzyme detection. Antibodies measured include IgM, IgA, IgG classes and IgG subclasses. As an alternative approach soluble clotting factor-specific antibodies are detected in the supernatants of PBMCs cultured in media alone for a couple of days (without any additional stimulants). ELISA or other methods commonly used for detecting soluble antibodies are then carried out to identify clotting factor-specific antibodies in the culture supernatants.

### Therapeutic Significance for the Patient

B-cells can produce antibodies at isolation from the body only when they are acutely engaged in immune processes (were recently activated in vivo). Therefore, detection of such B-cells can identify flares of anti-clotting factor-specific antibody production in vivo. Clinically, this implies that patients should be monitored to detect early on the development of therapy resistance in the subset of hemophilic patients who develop such. With each immunological relapse there is a likelihood of developing a more sever therapy resistance. Thus, the immune activity leading to the clinical therapy resistance can be monitored before the patient develops clinically evident therapy resistance. The direct B-cell assay of this invention therefore can advantageously be used as an immune monitoring tool with direct therapeutic implications for the patient. A positive direct B-cell assay response indicates that B cell inactivation treatment has to be initiated.

### Indirect Memory B-Cell Test

Memory B-cells require several days of stimulation with polyclonal activators before they start secreting antibodies. We therefore developed an in vitro “indirect B-cell test” or “indirect B-cell assay” to identify such cells from the blood. Polyclonal activation can be done in a pre-incubation step, in which PBMCs are stimulated with a B cell polyclonal activator such as CpG (bacterial DNA), an antigen of Staphylococcus aureaus Cowan, SAC, R848 being a TLR7/8 agonist that mimics contact of B cells to bacteria. Pokeweed mitogen (PWM), IL-2 (interleukin 2), IL-6, IL-10, and B-cell activating factor (BAFF) also known as tumor necrosis factor ligand superfamily member 13B (TNFL 13B), Anti-CD40 antibodies, and combinations thereof, including R848 plus IL-2 for 3-8 days in culture. Polyclonal activation may include pre-incubation with IL-2, R-848 and/or ß-mercaptoethanol and other protocols that are commonly used to polyclonally stimulate B-cells. FIG. 3 depicts a schematic strategy for carrying out an indirect B cell test.

### Approach

Peripheral blood mononuclear cells (PBMCs) are isolated from the blood, e.g. by density gradient centrifugation. The B cells contained in the sample are polyclonally stimulated e.g. with IL-2, and R-848 and ß-mercaptoethanol and subsequently transferred to filter plates for ELISPOT or FluoroSpot™ detection, or the supernatants of such polyclonally activated B cells are transferred into ELISA plates that have been coated with clotting factor antigen. The polyclonally activated B cells or their supernatants are then incubated on the plate. After suitable incubation time, allowing sufficient binding of secreted antibodies to the antigen on the plate, the clotting factor-specific antibodies are detected by either fluorescent or colorimetric detection. Antibodies measured include IgM, IgA IgG classes and IgG subclasses.

As an alternative approach soluble clotting factor-specific antibodies are detected in the supernatants of the polyclonally activated B cells using ELISA, or other methods commonly used for detecting soluble antibodies are then done to identify clotting factor-specific antibodies in the culture supernatants.

The indirect memory B-cell test shows that clotting factor-specific memory B-cells have become activated in vivo in haemophilia patients reflecting on the initial immune reactions towards developing therapy resistance. This finding has direct therapeutic consequences for the treatment of patients.

3) The magnitude of the positive test response in the direct and indirect memory B-cell tests is related to the magnitude of the clotting factor-specific B-cell response in individual patients, thus, should be proportionally related to the risk of developing resistance to clotting factor substitution therapy. Also, once either B-cell-specific or immune modulatory treatment is initiated to abrogate reduce the B cell mediated resistance to substitution therapy, the treatment success can be monitored via the indirect memory B-cell test. Treatment success is likely to be related to a diminution of the magnitude of the positive response in the indirect memory B-cell test (i.e., a decrease in spot number, spot size or intensity or the clotting factor-specific antibody concentration in the culture supernatant).

### ELISPOT Assays

According to embodiments of this invention, enzyme linked immune spot (“ELISPOT” assays can be carried out using methods described in U.S. Pat. Nos. 7,598,093 and 8,088,630, (incorporated herein fully by reference. Analyzers and methods from Cellular Technology Ltd (Immunospot® is a Registered Trademark of Cellular Technology Ltd. (“CTL”), Shaker Heights, OH). Analyzers and methods can be obtained from CTL.

### EXAMPLES

This invention is further illustrated by the following examples, which are not considered limiting to the scope of this invention. Rather, they are used to point out certain embodiments. Persons of skill in the art can use the descriptions and teachings herein to produce other embodiments that are within the scope of this invention. All such embodiments are considered to be part of this invention.

### Example 1: Detection of Clotting Factor-Specific B-Cells and Antibodies from Human Patients with Hemophilia Using a Direct B-Cell Test

### Methods:

Filter plates are coated with one of Factors I, II, V, VI, VI. VIII, IX, X, XI, XIII vWF, or Antithrombin III antigens. In some cases, Factor I alpha chain, Factor I beta chain, Factor I gamma chain Factor XIII alpha chain, Factor XIII beta chain or other portions of clotting factors chains can be used. PBMC are isolated from the blood by density gradient centrifugation and transferred to the plates as described in U.S. Pat. No. 7,598,093. After a suitable incubation time, allowing sufficient secretion and binding of antibodies to the coated antigen, the production of clotting factor-specific antibodies is detected by enzyme-linked immunoassay detection. Antibodies measured are primarily IgG.

### Results:

If we find that PBMCs isolated from patients with hemophilia contain plasma blasts that after isolation from the body actively produce detectable of Factors I, II, V. VII, VIII, IX, X, XI, XIII, vWF, or antithrombin III, and factor-specific IgG antibodies that suggests that resistance to substitution therapy is about to develop.

### Example 2: Detection of Clotting Factor-Specific Immune Cells from Human Patients with Haemophilia Using an Indirect B-Cell Test

Peripheral blood mononuclear cells (PBMCs) are isolated from the blood by density gradient centrifugation. The B-cells contained in the PBMC sample are polyclonally stimulated with IL-2, R-848 ß-mercaptoethanol for 4 days and subsequently transferred to filter plates coated with clotting factor antigen(s) (that is one of Factors I, II, VI, VII, VIII, IX, X, XI, XIII, vWF, or antithrombin III). The polyclonally activated cells are then incubated on the plate for 24 h. The production of clotting factor-specific antibodies and binding to the antigen on the plate is detected by colorimetric enzyme detection. Antibodies measured include IgG.

We find that PBMC of hemophilia patients stimulated with B-cell polyclonal activators produce anti-clotting factor antibodies, indicating that the B-cells from the patients react with clotting factors, indicating that the patient develops resistance to reconstitution therapy.

### Example 3: Treatment of Patients Having Haemophilia

Haemophilia A is characterized by a deficit in clotting Factor VIII. Haemophilia B is characterized by a deficit in Factor IX, and in a rare form, the deficit is in Factor XI. In normal subjects without haemophilia, the level of Factor VIII is in the normal range, depending on the specific assay for the Factor VIII. The level of Factor IX in patients without haemophilia is about 3 μg/ml, and the level of Factor XI is less than 5 μg/ml.

In patients with severe haemophilia A, Factor VIII is less than 1% of normal, and may be close to zero. In patients with moderate haemophilia A, the level of Factor VIII may be in the range of from 1-5% or normal. In mild cases of haemophilia A, the level of Factor VIII may be in the range of 5-40% of normal.

Treatment of haemophilia A can be accomplished by intravenous injection of Factor VIII. Examples of approved Factor VIII preparations include Helixate® FS, Recombinate®, Kogenate® FS. Advate®, ReFacto®, Eloctate®, Nuwiq®, Adynovate®, Kovaltry®, Jivi®, and Xyntha®. Human plasma-derived preparations include Monarc-M®, Monoclate-P®, Hemofil M®, and Koate-DVI®.

In patients with haemophilia B, the level of Factor IX is below about 3 μg/ml. In individuals with mild haemophilia B, Factor IX levels are generally between 5% and 40% of normal; those with moderate haemophilia B have Factor IX levels from 1 to 5% or normal, and those with severe haemophilia B have Factor IX levels below 1% of normal.

Replacement therapy with Factor IX may include use of the currently available recombinant Factor IX products: BeneFIX®, Rixubis®, Ixinity®, Alprolix®, Idelvion®, and Robinyn®.

In patients with Factor XI deficiency, Factor XI is below about 5% of 3-7 μg/ml, and replacement therapy is generally provided by intravenous injection of Factor XI.

In about 30% of individuals having severe clotting factor deficiency, patients may develop “inhibitors” against the replacement factors. The immune system may recognize a replacement factor as “foreign” and generate antibodies (inhibitors) which target and destroy the replacement factor. Inhibitor development can sometimes be accompanied by mild or serious allergic reactions.

The amount of such inhibitors is classified using so called Bethesda units. If the inhibitor titer is below 5 Bethesda units, the amount of replacement Factor needed can be still be provided by substitution. In patients with titers greater than 5 Bethesda units, simple replacement therapy may not be successful. In such cases, it may be possible to bypass the Factor VIII pathway, and instead inject Factor VII, Factor IIa, Factor VIIa, Factor IXa, or Factor Xa.

Treatment of resistance to replacement therapy by infusion of clotting factors I, II, V. VII, VIII, IX, X, XI, XIII, VWF, and antithrombin III can involve increasing the concentration of the clotting factor by about 1000-fold compared to the normal physiological concentration. One can pre-treat patients having B cell mediated resistance to replacement therapy with rituximab, starting with an infusion rate of about 100 mg/hr, and if no toxicity is observed, the dose can be increased every 30 minutes to a maximum of about 400 mg/hr.

Individuals exhibiting resistance to replacement therapy having developed antibodies against the injected clotting Factor(s) can be treated by infusing very high amounts of the clotting factor to induce specific B cell tolerance, i.e., sufficient to increase the in vivo amount of the factor by 1000-fold following general B cell depletion via rituximab injection.

We claim:

1. (canceled)

2. A method for detecting a B-cell antibody response to a clotting factor in a patient having haemophilia indicating the development of B cell mediated resistance to substitution therapy using an indirect B cell test, comprising:
a) adding a B-cell polyclonal activator to freshly isolated PBMCs for 3-8 days
b) providing a cell culture plate having a clotting factor antigen attached thereto;
c) placing a sample of polyclonally pre-stimulated PBMCs stimulated with one or more of CpG (bacterial DNA), an antigen of Staphylococcus aureaus Cowan, SAC, R848 being a TLR7/8 agonist that mimics contact of B cells to bacteria. Pokeweed mitogen (PWM), IL-2 (interleukin 2), IL-6, IL-10, and B-cell activating factor (BAFF) also known as tumor necrosis factor ligand superfamily member 13B (TNFL 13B), Anti-CD40 antibodies, and combinations thereof, including R848 plus IL-2 from said patient in cell culture medium into said cell culture plate of step b);
d) permitting B cells in said sample of PBMCs to produce antibodies, said antibodies binding to antigen of step b); and
e) detecting binding of said antibodies to said antigen.

3. A method for detecting a B-cell antibody response to a clotting factor in a patient having haemophilia indicating the development of B cell mediated resistance to substitution therapy using a direct ELISA test, comprising:
a) providing a sample of peripheral blood mononuclear cells (PBMCs) from said patient;
b) culturing these PBMC with cell culture medium alone for ˜1 day; and
c) testing supernatant by ELISA for clotting factor-specific antibodies.

4. A method for detecting a B-cell antibody response to a clotting factor in a patient having haemophilia with development of B cell mediated resistance to substitution therapy using an indirect ELISA test, comprising:
a) providing a sample of peripheral blood mononuclear cells (PBMCs) from said patient;
b) culturing these PBMCs with a polyclonal B cell activator for 3-8 days; and
c) testing supernatant by ELISA for clotting factor-specific antibodies.

5. A method for treating a patient having haemophilia and having resistance to replacement or substitution therapy caused by clotting factor-specific B cells in vivo comprising:
a) injection of rituximab at a starting does of about 100 mg/hr, and if no toxicity is observed, increasing the dose every 30 minutes up to a maximum dose of about 400 mg/hr; then
b) injecting intravenously, a dose of said clotting factor sufficient to produce about a 1,000-fold increase in in vivo concentration compared to the normal physiological concentration of the factor;
c) followed by daily injections of the same factor in a dose sufficient to maintain the high in vivo concentration for a duration of about 2 weeks.

6. The method of claim 5, where said injecting comprises injecting a clotting factor coupled with an immunotoxin (e.g., ricin) or immune suppressive agent to selectively kill or inactivate clotting factor-specific B cells.

7. The method of claim 6, further comprising assaying said patient's progress using an indirect B cell test.

8. The method of claim 7, wherein if the patient's resistance to substitution therapy for haemophilia A does not improve, inject one of the therapeutic agents selected from the group consisting of Helixate® FS, Recombinate®, Kogenate® FS, Advate®, ReFacto®, Eloctate®, Nuwiq®, Adynovate®, Kovaltry®, Jivi®, and Xyntha®. Human plasma-derived preparations include Monarc-M®, Monoclate-P®, Hemofil M®, and Koate-DVI®.

9. The method of claim 7, wherein if the patient's resistance to substitution therapy for haemophilia A does not improve, injecting one or more of Factor VII, or Factor IIa, or Factor VIIa, or Factor IXa, or Factor Xa.

10. The method of claim 7, wherein if the patient's resistance to substitution therapy for treatment of haemophilia B does not improve with injection of Factor IX, injecting one of the therapeutic agents selected from the group consisting of BeneFIX®, Rixubis®, Ixinity®, Alprolix®, Idelvion®, and Rebinyn®.

11. The method of any of claim 1, wherein said clotting factor is Factor I, or Factor II, or Factor V, or Factor VII, or Factor VIII, or Factor IX, or Factor X, or Factor XI, or Factor XIII, or von Willebrand Factor (VWF), or antithrombin III.

12. The method of any of claim 1, wherein detection of antibodies to clotting factors is accomplished using ELISPOT, ELISA, or FluoroSpot™ methods.


SEQUENCE LISTING (14 sequences)

SEQ ID NO: 1 - protein, 866 aa, Homo sapiens
MFSMRIVCLVLSVVGTAWTADSGEGDFLAEGGGVRGPRVVERHQSACKDSDWPFCSDEDWNYKCPSGCRMKGLIDEVNQDFTNRINKLKNSLFEYQKNNKDSHSLTTNIMEILRGDFSSANNRDNTYNRVSEDLRSRIEVLKRKVIEKVQHIQLLQKNVRAQLVDMKRLEVDIDIKIRSCRGSCSRALAREVDLKDYEDQQKQLEQVIAKDLLPSRDRQHLPLIKMKPVPDLVPGNFKSQLQKVPPEWKALTDMPQMRMELERPGGNEITRGGSTSYGTGSETESPRNPSSAGSWNSGSSGPGSTGNRNPGSSGTGGTATWKPGSSGPGSTGSWNSGSSGTGSTGNQNPGSPRPGSTGTWNPGSSERGSAGHWTSESSVSGSTGQWHSESGSFRPDSPGSGNARPNNPDWGTFEEVSGNVSPGTRREYHTEKLVTSKGDKELRTGKEKVTSGSTTTTRRSCSKTVTKTVIGPDGHKEVTKEVVTSEDGSDCPEAMDLGTLSGIGTLDGFRHRHPDEAAFFDTASTGKTFPGFFSPMLGEFVSETESRGSESGIFTNTKESSSHHPGIAEFPSRGKSSSYSKQFTSSTSYNRGDSTFESKSYKMADEAGSEADHEGTHSTKRGHAKSRPVRDCDDVLQTHPSGTQSGIFNIKLPGSSKIFSVYCDQETSLGGWLLIQQRMDGSLNFNRTWQDYKRGFGSLNDEGEGEFWLGNDYLHLLTQRGSVLRVELEDWAGNEAYAEYHFRVGSEAEGYALQVSSYEGTAGDALIEGSVEEGAEYTSHNNMQFSTFDRDADQWEENCAEVYGGGWWYNNCQAANLNGIYYPGGSYDPRNNSPYEIENGVVWVSFRGADYSLRAVRMKIRPLVTQ

SEQ ID NO: 2 - protein, 491 aa, Homo sapiens
MKRMVSWSFHKLKTMKHLLLLLLCVFLVKSQGVNDNEEGFFSARGHRPLDKKREEAPSLRPAPPPISGGGYRARPAKAAATQKKVERKAPDAGGCLHADPDLGVLCPTGCQLQEALLQQERPIRNSVDELNNNVEAVSQTSSSSFQYMYLLKDLWQKRQKQVKDNENVVNEYSSELEKHQLYIDETVNSNIPTNLRVLRSILENLRSKIQKLESDVSAQMEYCRTPCTVSCNIPVVSGKECEEIIRKGGETSEMYLIQPDSSVKPYRVYCDMNTENGGWTVIQNRQDGSVDFGRKWDPYKQGFGNVATNTDGKNYCGLPGEYWLGNDKISQLTRMGPTELLIEMEDWKGDKVKAHYGGFTVQNEANKYQISVNKYRGTAGNALMDGASQLMGENRTMTIHNGMFFSTYDRDNDGWLTSDPRKQCSKEDGGGWWYNRCHAANPNGRYYWGGQYTWDMAKHGTDDGVVWMNWKGSWYSMRKMSMKIRPFFPQQ

SEQ ID NO: 3 - protein, 453 aa, Homo sapiens
MSWSLHPRNLILYFYALLFLSSTCVAYVATRDNCCILDERFGSYCPTTCGIADFLSTYQTKVDKDLQSLEDILHQVENKTSEVKQLIKAIQLTYNPDESSKPNMIDAATLKSRKMLEEIMKYEASILTHDSSIRYLQEIYNSNNQKIVNLKEKVAQLEAQCQEPCKDTVQIHDITGKDCQDIANKGAKQSGLYFIKPLKANQQFLVYCEIDGSGNGWTVFQKRLDGSVDFKKNWIQYKEGFGHLSPTGTTEFWLGNEKIHLISTQSAIPYALRVELEDWNGRTSTADYAMFKVGPEADKYRLTYAYFAGGDAGDAFDGFDFGDDPSDKFFTSHNGMQFSTWDNDNDKFEGNCAEQDGSGWWMNKCHAGHLNGVYYQGGTYSKASTPNGYDNGIIWATWKTRWYSMKKTTMKIIPFNRLTIGEGQQHHLGGAKQVRPEHPAETEYDSLYPEDDL

SEQ ID NO: 4 - protein, 622 aa, Homo sapiens
MAHVRGLQLPGCLALAALCSLVHSQHVFLAPQQARSLLQRVRRANTFLEEVRKGNLERECVEETCSYEEAFEALESSTATDVFWAKYTACETARTPRDKLAACLEGNCAEGLGTNYRGHVNITRSGIECQLWRSRYPHKPEINSTTHPGADLQENFCRNPDSSTTGPWCYTTDPTVRRQECSIPVCGQDQVTVAMTPRSEGSSVNLSPPLEQCVPDRGQQYQGRLAVTTHGLPCLAWASAQAKALSKHQDFNSAVQLVENFCRNPDGDEEGVWCYVAGKPGDFGYCDLNYCEEAVEEETGDGLDEDSDRAIEGRTATSEYQTFFNPRTFGSGEADCGLRPLFEKKSLEDKTERELLESYIDGRIVEGSDAEIGMSPWQVMLFRKSPQELLCGASLISDRWVLTAAHCLLYPPWDKNFTENDLLVRIGKHSRTRYERNIEKISMLEKIYIHPRYNWRENLDRDIALMKLKKPVAFSDYIHPVCLPDRETAASLLQAGYKGRVTGWGNLKETWTANVGKGQPSVLQVVNLPIVERPVCKDSTRIRITDNMFCAGYKPDEGKRGDACEGDSGGPFVMKSPFNNRWYQMGIVSWGEGCDRDGKYGFYTHVFRLKKWIQKVIDQFGE

SEQ ID NO: 5 - protein, 2224 aa, Homo sapiens
MFPGCPRLWVLVVLGTSWVGWGSQGTEAAQLRQFYVAAQGISWSYRPEPTNSSLNLSVTSFKKIVYREYEPYFKKEKPQSTISGLLGPTLYAEVGDIIKVHFKNKADKPLSIHPQGIRYSKLSEGASYLDHTFPAEKMDDAVAPGREYTYEWSISEDSGPTHDDPPCLTHIYYSHENLIEDFNSGLIGPLLICKKGTLTEGGTQKTFDKQIVLLFAVFDESKSWSQSSSLMYTVNGYVNGTMPDITVCAHDHISWHLLGMSSGPELFSIHFNGQVLEQNHHKVSAITLVSATSTTANMTVGPEGKWIISSLTPKHLQAGMQAYIDIKNCPKKTRNLKKITREQRRHMKRWEYFIAAEEVIWDYAPVIPANMDKKYRSQHLDNFSNQIGKHYKKVMYTQYEDESFTKHTVNPNMKEDGILGPIIRAQVRDTLKIVFKNMASRPYSIYPHGVTFSPYEDEVNSSFTSGRNNTMIRAVQPGETYTYKWNILEFDEPTENDAQCLTRPYYSDVDIMRDIASGLIGLLLICKSRSLDRRGIQRAADIEQQAVFAVFDENKSWYLEDNINKFCENPDEVKRDDPKFYESNIMSTINGYVPESITTLGFCFDDTVQWHFCSVGTQNEILTIHFTGHSFIYGKRHEDTLTLFPMRGESVTVTMDNVGTWMLTSMNSSPRSKKLRLKFRDVKCIPDDDEDSYEIFEPPESTVMATRKMHDRLEPEDEESDADYDYQNRLAAALGIRSFRNSSLNQEEEEFNLTALALENGTEFVSSNTDIIVGSNYSSPSNISKFTVNNLAEPQKAPSHQQATTAGSPLRHLIGKNSVLNSSTAEHSSPYSEDPIEDPLQPDVTGIRLLSLGAGEFKSQEHAKHKGPKVERDQAAKHRFSWMKLLAHKVGRHLSQDTGSPSGMRPWEDLPSQDTGSPSRMRPWKDPPSDLLLLKQSNSSKILVGRWHLASEKGSYEIIQDTDEDTAVNNWLISPQNASRAWGESTPLANKPGKQSGHPKFPRVRHKSLQVRQDGGKSRLKKSQFLIKTRKKKKEKHTHHAPLSPRTFHPLRSEAYNTFSERRLKHSLVLHKSNETSLPTDLNQTLPSMDFGWIASLPDHNQNSSNDTGQASCPPGLYQTVPPEEHYQTFPIQDPDQMHSTSDPSHRSSSPELSEMLEYDRSHKSFPTDISQMSPSSEHEVWQTVISPDLSQVTLSPELSQTNLSPDLSHTTLSPELIQRNLSPALGQMPISPDLSHTTLSPDLSHTTLSLDLSQTNLSPELSQTNLSPALGQMPLSPDLSHTTLSLDFSQTNLSPELSHMTLSPELSQTNLSPALGQMPISPDLSHTTLSLDFSQTNLSPELSQTNLSPALGQMPLSPDPSHTTLSLDLSQTNLSPELSQTNLSPDLSEMPLFADLSQIPLTPDLDQMTLSPDLGETDLSPNFGQMSLSPDLSQVTLSPDISDTTLLPDLSQISPPPDLDQIFYPSESSQSLLLQEFNESFPYPDLGQMPSPSSPTLNDTFLSKEFNPLVIVGLSKDGTDYIEIIPKEEVQSSEDDYAEIDYVPYDDPYKTDVRTNINSSRDPDNIAAWYLRSNNGNRRNYYIAAEEISWDYSEFVQRETDIEDSDDIPEDTTYKKVVFRKYLDSTFTKRDPRGEYEEHLGILGPIIRAEVDDVIQVRFKNLASRPYSLHAHGLSYEKSSEGKTYEDDSPEWFKEDNAVQPNSSYTYVWHATERSGPESPGSACRAWAYYSAVNPEKDIHSGLIGPLLICQKGILHKDSNMPMDMREFVLLFMTFDEKKSWYYEKKSRSSWRLTSSEMKKSHEFHAINGMIYSLPGLKMYEQEWVRLHLLNIGGSQDIHVVHFHGQTLLENGNKQHQLGVWPLLPGSFKTLEMKASKPGWWLLNTEVGENQRAGMQTPFLIMDRDCRMPMGLSTGIISDSQIKASEFLGYWEPRLARLNNGGSYNAWSVEKLAAEFASKPWIQVDMQKEVIITGIQTQGAKHYLKSCYTTEFYVAYSSNQINWQIFKGNSTRNVMYFNGNSDASTIKENQFDPPIVARYIRISPTRAYNRPTLRLELQGCEVNGCSTPLGMENGKIENKQITASSFKKSWWGDYWEPFRARLNAQGRVNAWQAKANNNKQWLEIDLLKIKKITAIITQGCKSLSSEMYVKSYTIHYSEQGVEWKPYRLKSSMVDKIFEGNTNTKGHVKNFFNPPIISRFIRVIPKTWNQSIALRLELFGCDIY

SEQ ID NO: 6 - protein, 466 aa, Homo sapiens
MVSQALRLLCLLLGLQGCLAAGGVAKASGGETRDMPWKPGPHRVFVTQEEAHGVLHRRRRANAFLEELRPGSLERECKEEQCSFEEAREIFKDAERTKLFWISYSDGDQCASSPCQNGGSCKDQLQSYICFCLPAFEGRNCETHKDDQLICVNENGGCEQYCSDHTGTKRSCRCHEGYSLLADGVSCTPTVEYPCGKIPILEKRNASKPQGRIVGGKVCPKGECPWQVLLLVNGAQLCGGTLINTIWVVSAAHCFDKIKNWRNLIAVLGEHDLSEHDGDEQSRRVAQVIIPSTYVPGTTNHDIALLRLHQPVVLTDHVVPLCLPERTFSERTLAFVRFSLVSGWGQLLDRGATALELMVLNVPRLMTQDCLQQSRKVGDSPNITEYMFCAGYSDGSKDSCKGDSGGPHATHYRGTWYLTGIVSWGQGCATVGHFGVYTRVSQYIEWLQKLMRSEPRPGVLLRAPFP

SEQ ID NO: 7 - protein, 2351 aa, Homo sapiens
MQIELSTCFFLCLLRFCFSATRRYYLGAVELSWDYMQSDLGELPVDARFPPRVPKSFPFNTSVVYKKTLFVEFTDHLFNIAKPRPPWMGLLGPTIQAEVYDTVVITLKNMASHPVSLHAVGVSYWKASEGAEYDDQTSQREKEDDKVFPGGSHTYVWQVLKENGPMASDPLCLTYSYLSHVDLVKDLNSGLIGALLVCREGSLAKEKTQTLHKFILLFAVFDEGKSWHSETKNSLMQDRDAASARAWPKMHTVNGYVNRSLPGLIGCHRKSVYWHVIGMGTTPEVHSIFLEGHTFLVRNHRQASLEISPITFLTAQTLLMDLGQFLLFCHISSHQHDGMEAYVKVDSCPEEPQLRMKNNEEAEDYDDDLTDSEMDVVRFDDDNSPSFIQIRSVAKKHPKTWVHYIAAEEEDWDYAPLVLAPDDRSYKSQYLNNGPQRIGRKYKKVRFMAYTDETFKTREAIQHESGILGPLLYGEVGDTLLIIFKNQASRPYNIYPHGITDVRPLYSRRLPKGVKHLKDFPILPGEIFKYKWTVTVEDGPTKSDPRCLTRYYSSFVNMERDLASGLIGPLLICYKESVDQRGNQIMSDKRNVILFSVFDENRSWYLTENIQRFLPNPAGVQLEDPEFQASNIMHSINGYVFDSLQLSVCLHEVAYWYILSIGAQTDFLSVFFSGYTFKHKMVYEDTLTLFPFSGETVFMSMENPGLWILGCHNSDFRNRGMTALLKVSSCDKNTGDYYEDSYEDISAYLLSKNNAIEPRSFSQNSRHPSTRQKQFNATTIPENDIEKTDPWFAHRTPMPKIQNVSSSDLLMLLRQSPTPHGLSLSDLQEAKYETFSDDPSPGAIDSNNSLSEMTHFRPQLHHSGDMVFTPESGLQLRLNEKLGTTAATELKKLDFKVSSTSNNLISTIPSDNLAAGTDNTSSLGPPSMPVHYDSQLDTTLFGKKSSPLTESGGPLSLSEENNDSKLLESGLMNSQESSWGKNVSSTESGRLFKGKRAHGPALLTKDNALFKVSISLLKTNKTSNNSATNRKTHIDGPSLLIENSPSVWQNILESDTEFKKVTPLIHDRMLMDKNATALRLNHMSNKTTSSKNMEMVQQKKEGPIPPDAQNPDMSFFKMLFLPESARWIQRTHGKNSLNSGQGPSPKQLVSLGPEKSVEGQNFLSEKNKVVVGKGEFTKDVGLKEMVFPSSRNLFLTNLDNLHENNTHNQEKKIQEEIEKKETLIQENVVLPQIHTVTGTKNFMKNLFLLSTRQNVEGSYDGAYAPVLQDFRSLNDSTNRTKKHTAHFSKKGEEENLEGLGNQTKQIVEKYACTTRISPNTSQQNFVTQRSKRALKQFRLPLEETELEKRIIVDDTSTQWSKNMKHLTPSTLTQIDYNEKEKGAITQSPLSDCLTRSHSIPQANRSPLPIAKVSSFPSIRPIYLTRVLFQDNSSHLPAASYRKKDSGVQESSHFLQGAKKNNLSLAILTLEMTGDQREVGSLGTSATNSVTYKKVENTVLPKPDLPKTSGKVELLPKVHIYQKDLFPTETSNGSPGHLDLVEGSLLQGTEGAIKWNEANRPGKVPFLRVATESSAKTPSKLLDPLAWDNHYGTQIPKEEWKSQEKSPEKTAFKKKDTILSLNACESNHAIAAINEGQNKPEIEVTWAKQGRTERLCSQNPPVLKRHQREITRTTLQSDQEEIDYDDTISVEMKKEDFDIYDEDENQSPRSFQKKTRHYFIAAVERLWDYGMSSSPHVLRNRAQSGSVPQFKKVVFQEFTDGSFTQPLYRGELNEHLGLLGPYIRAEVEDNIMVTFRNQASRPYSFYSSLISYEEDQRQGAEPRKNFVKPNETKTYFWKVQHHMAPTKDEFDCKAWAYFSDVDLEKDVHSGLIGPLLVCHTNTLNPAHGRQVTVQEFALFFTIFDETKSWYFTENMERNCRAPCNIQMEDPTFKENYRFHAINGYIMDTLPGLVMAQDQRIRWYLLSMGSNENIHSIHFSGHVFTVRKKEEYKMALYNLYPGVFETVEMLPSKAGIWRVECLIGEHLHAGMSTLFLVYSNKCQTPLGMASGHIRDFQITASGQYGQWAPKLARLHYSGSINAWSTKEPFSWIKVDLLAPMIIHGIKTQGARQKFSSLYISQFIIMYSLDGKKWQTYRGNSTGTLMVFFGNVDSSGIKHNIFNPPIIARYIRLHPTHYSIRSTLRMELMGCDLNSCSMPLGMESKAISDAQITASSYFTNMFATWSPSKARLHLQGRSNAWRPQVNNPKEWLQVDFQKTMKVTGVTTQGVKSLLTSMYVKEFLISSSQDGHQWTLFFQNGKVKVFQGNQDSFTPVVNSLDPPLLTRYLRIHPQSWVHQIALRMEVLGCEAQDLY

SEQ ID NO: 8 - protein, 461 aa, Homo sapiens
MQRVNMIMAESPGLITICLLGYLLSAECTVFLDHENANKILNRPKRYNSGKLEEFVQGNLERECMEEKCSFEEAREVFENTERTTEFWKQYVDGDQCESNPCLNGGSCKDDINSYECWCPFGFEGKNCELDVTCNIKNGRCEQFCKNSADNKVVCSCTEGYRLAENQKSCEPAVPFPCGRVSVSQTSKLTRAETVFPDVDYVNSTEAETILDNITQSTQSFNDFTRVVGGEDAKPGQFPWQVVLNGKVDAFCGGSIVNEKWIVTAAHCVETGVKITVVAGEHNIEETEHTEQKRNVIRIIPHHNYNAAINKYNHDIALLELDEPLVLNSYVTPICIADKEYTNIFLKFGSGYVSGWGRVFHKGRSALVLQYLRVPLVDRATCLRSTKFTIYNNMFCAGFHEGGRDSCQGDSGGPHVTEVEGTSFLTGIISWGEECAMKGKYGIYTKVSRYVNWIKEKTKLT

SEQ ID NO: 9 - protein, 488 aa, Homo sapiens
MGRPLHLVLLSASLAGLLLLGESLFIRREQANNILARVTRANSFLEEMKKGHLERECMEETCSYEEAREVFEDSDKTNEFWNKYKDGDQCETSPCQNQGKCKDGLGEYTCTCLEGFEGKNCELFTRKLCSLDNGDCDQFCHEEQNSVVCSCARGYTLADNGKACIPTGPYPCGKQTLERRKRSVAQATSSSGEAPDSITWKPYDAADLDPTENPFDLLDFNQTQPERGDNNLTRIVGGQECKDGECPWQALLINEENEGFCGGTILSEFYILTAAHCLYQAKRFKVRVGDRNTEQEEGGEAVHEVEVVIKHNRFTKETYDFDIAVLRLKTPITFRMNVAPACLPERDWAESTLMTQKTGIVSGFGRTHEKGRQSTRLKMLEVPYVDRNSCKLSSSFIITQNMFCAGYDTKQEDACQGDSGGPHVTRFKDTYFVTGIVSWGEGCARKGKYGIYTKVTAFLKWIDRSMKTRGLPKAKSHAPEVITSSPLK

SEQ ID NO: 10 - protein, 568 aa, Homo sapiens
MIFLYQVVHFILFTSVSGECVTQLLKDTCFEGGDITTVFTPSAKYCQVVCTYHPRCLLFTFTAESPSEDPTRWFTCVLKDSVTETLPRVNRTAAISGYSFKQCSHQISACNKDIYVDLDMKGINYNSSVAKSAQECQERCTDDVHCHFFTYATRQFPSLEHRNICLLKHTQTGTPTRITKLDKVVSGFSLKSCALSNLACIRDIFPNTVFADSNIDSVMAPDAFVCGRICTHHPGCLFFTFFSQEWPKESQRNLCLLKTSESGLPSTRIKKSKALSGFSLQSCRHSIPVFCHSSFYHDTDFLGEELDIVAAKSHEACQKLCTNAVRCQFFTYTPAQASCNEGKGKCYLKLSSNGSPTKILHGRGGISGYTLRLCKMDNECTTKIKPRIVGGTASVRGEWPWQVTLHTTSPTQRHLCGGSIIGNQWILTAAHCFYGVESPKILRVYSGILNQSEIKEDTSFFGVQEIIIHDQYKMAESGYDIALTNEECQKRYRGHKITHKMICAGYREGGKDACKGDSGGPLSCKHNEVWHLVGITSWGEGCAQRERPGVYTNVVEYVDWILEKTQAV

SEQ ID NO: 11 - protein, 732 aa, Homo sapiens [sequence omitted]

SEQ ID NO: 12 - protein, 661 aa, Homo sapiens
MRLKNLTFIIILIISGELYAEEKPCGFPHVENGRIAQYYYTFKSFYFPMSIDKKLSFFCLAGYTTESGRQEEQTTCTTEGWSPEPRCFKKCTKPDLSNGYISDVKLLYKIQENMRYGCASGYKTTGGKDEEVVQCLSDGWSSQPTCRKEHETCLAPELYNGNYSTTQKTFKVKDKVQYECATGYYTAGGKKTEEVECLTYGWSLTPKCTKLKCSSLRLIENGYFHPVKQTYEEGDVVQFFCHENYYLSGSDLIQCYNFGWYPESPVCEGRRNRCPPPPLPINSKIQTHSTTYRHGEIVHIECELNFEIHGSAEIRCEDGKWTEPPKCIEGQEKVACEEPPFIENGAANLHSKIYYNGDKVTYACKSGYLLHGSNEITCNRGKWTLPPECVENNENCKHPPVVMNGAVADGILASYATGSSVEYRCNEYYLLRGSKISRCEQGKWSSPPVCLEPCTVNVDYMNRNNIEMKWKYEGKVLHGDLIDFVCKQGYDLSPLTPLSELSVQCNRGEVKYPLCTRKESKGMCTSPPLIKHGVIISSTVDTYENGSSVEYRCFDHHFLEGSREAYCLDGMWTTPPLCLEPCTLSFTEMEKNNLLLKWDFDNRPHILHGEYIEFICRGDTYPAELYITGSILRMQCDRGQLKYPRCIPRQSTLSYQEPLRT

SEQ ID NO: 13 - protein, 2813 aa, Homo sapiens
MIPARFAGVLLALALILPGTLCAEGTRGRSSTARCSLFGSDFVNTFDGSMYSFAGYCSYLLAGGCQKRSFSIIGDFQNGKRVSLSVYLGEFFDIHLFVNGTVTQGDQRVSMPYASKGLYLETEAGYYKLSGEAYGFVARIDGSGNFQVLLSDRYFNKTCGLCGNFNIFAEDDFMTQEGTLTSDPYDFANSWALSSGEQWCERASPPSSSCNISSGEMQKGLWEQCQLLKSTSVFARCHPLVDPEPFVALCEKTLCECAGGLECACPALLEYARTCAQEGMVLYGWTDHSACSPVCPAGMEYRQCVSPCARTCQSLHINEMCQERCVDGCSCPEGQLLDEGLCVESTECPCVHSGKRYPPGTSLSRDCNTCICRNSQWICSNEECPGECLVTGQSHFKSFDNRYFTFSGICQYLLARDCQDHSFSIVIETVQCADDRDAVCTRSVTVRLPGLHNSLVKLKHGAGVAMDGQDVQLPLLKGDLRIQHTVTASVRLSYGEDLQMDWDGRGRLLVKLSPVYAGKTCGLCGNYNGNQGDDFLTPSGLAEPRVEDFGNAWKLHGDCQDLQKQHSDPCALNPRMTRFSEEACAVLTSPTFEACHRAVSPLPYLRNCRYDVCSCSDGRECLCGALASYAAACAGRGVRVAWREPGRCELNCPKGQVYLQCGTPCNLTCRSLSYPDEECNEACLEGCFCPPGLYMDERGDCVPKAQCPCYYDGEIFQPEDIFSDHHTMCYCEDGFMHCTMSGVPGSLLPDAVLSSPLSHRSKRSLSCRPPMVKLVCPADNLRAEGLECTKTCQNYDLECMSMGCVSGCLCPPGMVRHENRCVALERCPCFHQGKEYAPGETVKIGCNTCVCQDRKWNCTDHVCDATCSTIGMAHYLTFDGLKYLFPGECQYVLVQDYCGSNPGTFRILVGNKGCSHPSVKCKKRVTILVEGGEIELFDGEVNVKRPMKDETHFEVVESGRYIILLLGKALSVVWDRHLSISVVLKQTYQEKVCGLCGNFDGIQNNDLTSSNLQVEEDPVDFGNSWKVSSQCADTRKVPLDSSPATCHNNIMKQTMVDSSCRILTSDVFQDCNKLVDPEPYLDVCIYDTCSCESIGDCACFCDTIAAYAHVCAQHGKVVTWRTATLCPQSCEERNLRENGYECEWRYNSCAPACQVTCQHPEPLACPVQCVEGCHAHCPPGKILDELLQTCVDPEDCPVCEVAGRRFASGKKVTLNPSDPEHCQICHCDVVNLTCEACQEPGGLVVPPTDAPVSPTTLYVEDISEPPLHDFYCSRLLDLVFLLDGSSRLSEAEFEVLKAFVVDMMERLRISQKWVRVAVVEYHDGSHAYIGLKDRKRPSELRRIASQVKYAGSQVASTSEVLKYTLFQIFSKIDRPEASRITLLLMASQEPQRMSRNFVRYVQGLKKKKVIVIPVGIGPHANLKQIRLIEKQAPENKAFVLSSVDELEQQRDEIVSYLCDLAPEAPPPTLPPDMAQVTVGPGLLGVSTLGPKRNSMVLDVAFVLEGSDKIGEADFNRSKEFMEEVIQRMDVGQDSIHVTVLQYSYMVTVEYPFSEAQSKGDILQRVREIRYQGGNRTNTGLALRYLSDHSFLVSQGDREQAPNLVYMVTGNPASDEIKRLPGDIQVVPIGVGPNANVQELERIGWPNAPILIQDFETLPREAPDLVLQRCCSGEGLQIPTLSPAPDCSQPLDVILLLDGSSSFPASYFDEMKSFAKAFISKANIGPRLTQVSVLQYGSITTIDVPWNVVPEKAHLLSLVDVMQREGGPSQIGDALGFAVRYLTSEMHGARPGASKAVVILVTDVSVDSVDAAADAARSNRVTVFPIGIGDRYDAAQLRILAGPAGDSNVVKLQRIEDLPTMVTLGNSFLHKLCSGFVRICMDEDGNEKRPGDVWTLPDQCHTVTCQPDGQTLLKSHRVNCDRGLRPSCPNSQSPVKVEETCGCRWTCPCVCTGSSTRHIVTFDGQNFKLTGSCSYVLFQNKEQDLEVILHNGACSPGARQGCMKSIEVKHSALSVELHSDMEVTVNGRLVSVPYVGGNMEVNVYGAIMHEVRFNHLGHIFTFTPQNNEFQLQLSPKTFASKTYGLCGICDENGANDFMLRDGTVTTDWKTLVQEWTVQRPGQTCQPILEEQCLVPDSSHCQVLLLPLFAECHKVLAPATFYAICQQDSCHQEQVCEVIASYAHLCRTNGVCVDWRTPDFCAMSCPPSLVYNHCEHGCPRHCDGNVSSCGDHPSEGCFCPPDKVMLEGSCVPEEACTQCIGEDGVQHQFLEAWVPDHQPCQICTCLSGRKVNCTTQPCPTAKAPTCGLCEVARLRQNADQCCPEYECVCDPVSCDLPPVPHCERGLQPTLTNPGECRPNFTCACRKEECKRVSPPSCPPHRLPTLRKTQCCDEYECACNCVNSTVSCPLGYLASTATNDCGCTTTTCLPDKVCVHRSTIYPVGQFWEEGCDVCTCTDMEDAVMGLRVAQCSQKPCEDSCRSGFTYVLHEGECCGRCLPSACEVVTGSPRGDSQSSWKSVGSQWASPENPCLINECVRVKEEVFIQQRNVSCPQLEVPVCPSGFQLSCKTSACCPSCRCERMEACMLNGTVIGPGKTVMIDVCTTCRCMVQVGVISGFKLECRKTTCNPCPLGYKEENNTGECCGRCLPTACTIQLRGGQIMTLKRDETLQDGCDTHFCKVNERGEYFWEKRVTGCPPFDEHKCLAEGGKIMKIPGTCCDTCEEPECNDITARLQYVKVGSCKSEVEVDIHYCQGKCASKAMYSIDINDVQDQCSCCSPTRTEPMQVALHCTNGSVVYHEVLNAMECKCSPRKCSK

SEQ ID NO: 14 - protein, 464 aa, Homo sapiens
MYSNVIGTVTSGKRKVYLLSLLLIGFWDCVTCHGSPVDICTAKPRDIPMNPMCIYRSPEKKATEDEGSEQKIPEATNRRVWELSKANSRFATTFYQHLADSKNDNDNIFLSPLSISTAFAMTKLGACNDTLQQLMEVFKFDTISEKTSDQIHFFFAKLNCRLYRKANKSSKLVSANRLFGDKSLTFNETYQDISELVYGAKLQPLDFKENAEQSRAAINKWVSNKTEGRITDVIPSEAINELTVLVLVNTIYFKGLWKSKFSPENTRKELFYKADGESCSASMMYQEGKFRYRRVAEGTQVLELPFKGDDITMVLILPKPEKSLAKVEKELTPEVLQEWLDELEEMMLVVHMPRFRIEDGFSLKEQLQDMGLVDLFSPEKSKLPGIVAEGRDDLYVSDAFHKAFLEVNEEGSEAAASTAVVIAGRSLNPNRVTFKANRPFLVFIREVPLNTIIFMGRVANPCVK